(19) United States
(12) Patent Application Publication
(10) Pub. No.: US 2026/0325160 A1
(43) Pub. Date: Oct. 22, 2026

(54) HYDRO DISSECTION, IRRIGATION, AND SUCTION ELECTROCAUTERY DEVICES AND METHODS OF USE

(71) Applicant: Dinesh Vyas, Elk Grove, CA (US)

(72) Inventors: Dinesh Vyas, Elk Grove, CA (US); Jeffrey J. Christian, Morgan Hill, CA (US)

(21) Appl. No.: 19/642,500

(22) Filed: Apr. 8, 2026

Related U.S. Application Data
(63) Continuation of application No. PCT/US24/50604, filed on Oct. 9, 2024.
(63) Continuation-in-part of application No. 18/378,354, filed on Oct. 10, 2023.

(51) Int. Cl. A61B 17/3203; A61B 17/00; A61B 18/00; A61B 18/12; A61B 18/14

(52) U.S. Cl. CPC A61B 17/32037; A61B 18/1206; A61B 18/1445; A61B 2017/00336; A61B 2017/00367; A61B 2017/00876; A61B 2018/00595; A61B 2018/00922; A61B 2018/1452; A61B 2217/005; A61B 2217/007

(57) ABSTRACT

A laparoscopic instrument integrates electrocautery forceps with high velocity hydro-dissection, low velocity irrigation, and suction capability. When a sheath containing a distal internal bushing is fully retracted on the forceps shaft, exposing its jaws, the inner lumen of the bushing seals against the outer surface of the forceps shaft. Fluid inside the sheath is directed to a tiny nozzle, forming a high velocity fluid jet for tissue hydro-dissection. An electrode attached to the sheath retraction actuator makes electrical contact with a corresponding electrode in the device handle housing to deliver electrocautery only when the forceps are extended. Magnetic coupling ensures stable electrical contact and provides user haptic feedback regarding full sheath retraction. Distal sheath extension enlarges the flow path between the tapered forceps jaws and the inner lumen of the bushing, providing low velocity fluid irrigation or allowing fluid suction to occur.

### RELATED APPLICATION DATA

The present application is a continuation of co-pending International Application No. PCT/US2024/050604, filed Oct. 9, 2024, is a continuation-in-part of co-pending U.S. application Ser. No. 18/378,354, filed Oct. 10, 2023, and is related to U.S. application Ser. No. 18/089,368, filed Dec. 27, 2022, which claims benefit of U.S. provisional applications Serial Nos. 63/312,770, filed Feb. 22, 2022, and 63/421,511, filed Nov. 1, 2022, the entire disclosures of which are expressly incorporated by reference herein the entire disclosure of which is expressly incorporated by reference herein.

### TECHNICAL FIELD

The present application relates to surgical devices and systems and, more particularly, to devices and methods for tissue dissection, e.g., conducted during laparoscopic surgery, which may involve blunt tissue dissection performed via a focused fluid jet instead of the mechanical tissue disruption typically performed by laparoscopic dissectors and graspers.

For example, the devices and systems herein may include a laparoscopic instrument that includes modes of mechanical tissue dissection, hydro dissection, suction, electrocautery, and irrigation provided in a single instrument.

### BACKGROUND

In laparoscopic, thoracic, gynecological, urological, and minimally invasive surgery, such as robotic surgical procedures, isolation of anatomic structures, such as blood vessels and ducts, are performed via blunt dissection maneuvers involving spreading and tearing of soft tissue adjacent to the vessels and ducts. If the organ involved in the surgery is ischemic or necrotic, the organ and surrounding soft tissue becomes swollen and edematous, making it impossible to discern the outlines and locations of underlying ducts and vessels. Surgical maneuvers with existing laparoscopic graspers and dissectors carry significant potential of disrupting unrecognized organs, ducts, and vessels, which may lead to spillage of toxic infective contents into the abdominal cavity and hemorrhage.

Blunt dissection of soft tissue during laparoscopic surgery may be hazardous when the tissue is swollen and edematous, and the outline of blood vessels and ducts coursing through the soft tissue is not visible via endoscopic observation. In gangrenous cholecystitis, the gallbladder is distended to such a degree that it becomes ischemic, with compromise to its blood supply. Necrosis of the organ occurs, and the resultant inflammation and tissue swelling in the area of the gallbladder obscures the location of the cystic duct and the cystic artery. These structures must be surgically isolated, ligated or clipped, and transected for gallbladder removal. Mechanical dissection of the gangrenous gallbladder with traditional laparoscopic instruments, such as Maryland dissectors, may easily cause transection or laceration of non-visualized common bile duct, portal vein, colon, other intestines, cystic duct, and cystic artery, causing spillage of infected bile in the abdominal cavity and hemorrhage.

Other endoscopic procedures requiring execution of difficult and hazardous surgical blunt dissection include intra-abdominal endometriosis lesion resection, adhesiolysis or lysis of adhesions, and video assisted thoracic surgery or VATS, generally involving lung resection procedures and drainage of empyema.

Previous laparoscopic forceps exist that supply fluid irrigation and suction to the jaws of the instruments. One such instrument, described by Fischer in U.S. Pat. No. 9,308,014, teaches the use of a fluid jet at or in a stationary jaw of a forceps to dissect tissue.

Therefore, improved devices and methods for performing dissection of tissue within a patient's body would be useful.

### SUMMARY

The present application relates generally to surgical devices and systems, and, more particularly, to devices for tissue dissection, e.g., conducted in laparoscopic and/or robotic surgery, and to systems and methods for using such devices. The devices may include surgical instruments, e.g., a laparoscopic grasper, forceps, scissors, clip applier, vessel sealer, the like, that provide multiple modes of operation, e.g., hydro dissection, suction, and/or irrigation in addition to other optional mechanical functionalities, e.g., including end effectors for mechanical tissue dissection, cutting, and the like, provided in a single instrument. The devices may be provided in systems that include other components for operating the devices, e.g., sources or fluid and/or vacuum, electrical power sources, and the like. The devices may also be included in robotic surgical systems that may be operated remotely.

In one example, a surgical instrument or device is provided, e.g., a dissector forceps instrument for laparoscopic surgery, that includes mechanical tissue dissection, electrocautery, hydro dissection, suction and irrigation in a single instrument. Tissue manipulation, blunt tissue dissection and electrocautery may be conducted with the jaws of the laparoscopic forceps exposed distal to a tip of a retracted coaxial sheath. With the instrument in this configuration, the shaft of the forceps occupies the central lumen of a bushing located at the distal tip of the coaxial sheath, forcing all fluid flow to exit a small diameter nozzle in the bushing to create a high velocity fluid jet. Tissue hydro dissection may be performed in this configuration, with the forceps grasping tissue to provide counter-traction while the high velocity fluid jet performs atraumatic surgical dissection without the mechanical tissue disruption typically performed by laparoscopic dissectors and graspers. Hydro dissection safely isolates and dissects ducts, blood vessels and other anatomic structures during surgical procedures, e.g., in gangrenous or edematous tissue and organs.

The coaxial sheath of the instrument may be extended distally to cover the jaws of the forceps. As the cross-sectional area of the tapered jaws is less than the cross-sectional area of the shaft, an enlarged fluid path is formed at the tip of the outer sheath to enable suctioning, or to provide low velocity fluid irrigation. Suction may be used to clear hydro dissection fluid and/or blood from the surgical field, and fluid irrigation may be used to clear the suction cannula from clogging, e.g., due to tissue debris and/or blood clots. In one example, the instrument may include a self-contained battery operated fluid pump and/or a port that allows device connection to a vacuum cannister in the operating room.

In one example, the jaws of the forceps are electrically conductive, and electrocautery energy may be supplied to them by connecting the instrument to an electrosurgical generator. The jaws may cauterize and/or cut tissue when they are fully exposed out of the retracted coaxial sheath. When the coaxial sheath is extended distally to cover the jaws, the conductive path that supplies electrocautery energy is disengaged to prevent inadvertent activation of the forceps jaw tips in the irrigation and suction modes, which may otherwise potentially cause electrical arcing to tissue in the vicinity of the outer sheath tip that may injure both the tissue and the sheath tip.

In one example, electrocautery engagement and disengagement may be performed via an electrical switch incorporated into the sliding actuator that controls coaxial sheath extension and retraction. For example, coupled magnets in the sliding actuator and the instrument handle housing may be provided in proximity to the electrodes that comprise the electrical switch. These magnets may lock the electrodes together to ensure a stable electrical connection. Optionally, during retraction of the sheath to activate the electrical switch, the magnets may also create an impulse upon repositioning that provides haptic and aural feedback to the surgeon indicating that full sheath retraction and electrocautery connection has occurred.

In a particular example, a hydro dissection, irrigation and suction laparoscopic forceps instrument may be provided that includes a laparoscopic forceps including an elongate shaft, e.g., having an outer diameter of about three millimeters (3 mm) or smaller, that resides inside a coaxial sheath, e.g., having a relatively thin wall and an outer diameter of about five millimeters (5 mm). The coaxial sheath extends nearly the entire length of the shaft of the forceps, and may translate axially along the forceps shaft, e.g., between a proximal position where jaws on the distal end of the shaft are fully exposed and distal position where the coaxial sheath covers or encloses the jaws.

In one example, axial translation of the coaxial sheath is performed via an actuator located on a handle on a proximal end of the instrument. For example, a bushing may be attached to the distal tip of the coaxial sheath, e.g., having a length of about twelve millimeters (12 mm) and including a lumen having an inner diameter of about four millimeters (4 mm). The bushing may include an end cap, e.g., having a thickness of about one millimeter (1 mm) that includes a central lumen with an inner diameter that provides a sliding fit with the outer diameter of the shaft of the forceps and a tiny offset lumen, e.g., having an inner diameter of about 0.3 millimeters (0.3 mm). When the coaxial sheath is fully retracted, the central lumen of the end cap on the bushing seals against the outer surface of the forceps shaft, and all fluid flow then exits the offset lumen, forming a high velocity fluid jet. When the coaxial sheath is fully extended to enclose the forceps jaws, injected fluid flows through a low resistance path, i.e., with ample clearance between the forceps shaft and the inner surface of the coaxial sheath, and between the outer surface of the jaws and the central lumen of the bushing end cap, to produce low velocity fluid irrigation, or to allow vacuum suction to occur.

In one example, fluid flow is provided via a battery powered fluid pump integrated into the handle, with a fluid line connecting the pump to a fluid supply, e.g., an elevated intravenous saline bag. A separate connector in the handle may be coupled to a vacuum line, e.g., a standard operating room vacuum source. An actuator may be provided, e.g., a specialized spring loaded three-way control valve, may be included on the device handle. For example, a three-way valve may be provided that is movable between two positions by a control button or other actuator, e.g., a first position to couple the pump to the lumen of coaxial sheath to provide fluid flow or a second position to couple the vacuum to the lumen to provide aspiration. In one example, the actuator is biased to a normal resting position, where the valve permits fluid flow to occur. When the control button is pressed, the valve shuttles to the second position, cutting off fluid flow while opening the vacuum line.

In one example, fluid flow is provided by a battery powered electric pump, e.g., activated by depression of an electrical button switch in the handle. For example, two nine volt batteries may supply power to the pump via an electronic control board that maintains a constant voltage delivery to ensure that a constant fluid jet velocity is achieved for reliable tissue hydro dissection. Optionally, a rotational switch or other actuator may be provided that controls a potentiometer may also be provided on the handle to allow a user to adjust the velocity of the hydro dissection jet.

Optionally, the laparoscopic forceps may include electrocautery capability, and a connector on the handle of the device may interface with a power cable attached to an electrosurgical generator. The electrosurgical generator may be configured to supply electricall energy, e.g., radiofrequency energy, to the forceps jaws, allowing the jaws to cauterize or cut tissue in the surgical field. Activation of electrocautery energy is enabled only when the forceps jaws are fully exposed in their functional mode, with the coaxial sheath fully retracted, to avoid inadvertent electrical discharge to internal organs and tissue, with concomitant thermal destruction of the distal tip of the extended sheath.

Optionally, any of the devices and systems herein may include one or more additional components or functionalities. For example, the devices may include one or more sensors on or adjacent their end effectors, e.g., Doppler or other sensors for identifying blood flow in contacted tissue, microfluidics sensors for identifying tissue characteristics and the like, one or more electrodes or other cautery elements, vessel sealing elements, and/or one or more imaging elements. One or more processors or controllers may be coupled to these elements, e.g., to analyze signals from sensors, generate images on a display, and the like. If one or more imaging elements are provided on the devices, a display may be provided, e.g., mounted on a proximal end of the devices or remotely from the devices, which may allow visual monitoring of a surgical field during use of the devices.

In accordance with one example, an instrument, e.g., a laparoscopic grasper or forceps, is provided that includes double action jaws that include rigid nozzles on lateral aspects of each jaw. Fluid-carrying channels extend along lateral aspects of the instrument shaft, and a length of flexible hose connects the distal end of each channel to a proximal end of a respective jaw nozzle channel. The short flexible hose sections may adopt a substantially straightened position when the jaws are closed, such that their outer profile does not exceed the outer profile of the instrument shaft, allowing the instrument to be inserted through a trocar, e.g., a five millimeter (5 mm) laparoscopic trocar.

In another example, a laparoscopic forceps is provided that includes multiple modes of operation, e.g., including two or more of mechanical tissue dissection, hydro dissection, suction and irrigation, cautery, vessel sealing, and microfludics provided in a single instrument. Alternatively, other surgical instruments may be provided that include these modes of operation, e.g., a bowel grasper, clip applier, scissors, vessel sealer, and the like. Tissue manipulation and blunt tissue dissection may be conducted with jaws of the forceps extended distal to a tip of a coaxial sheath. Tissue hydro dissection may also be performed in this configuration, with the forceps grasping tissue to provide counter-traction while a high velocity fluid jet performs atraumatic surgical dissection without the mechanical tissue disruption typically performed by conventional laparoscopic dissectors and graspers. Hydro dissection may safely isolate and/or dissect ducts, blood vessels and other anatomic structures during surgical procedures in gangrenous or edematous tissue and organs. The forceps may be retracted fully into the sheath, e.g., to perform suctioning and/or to perform pure hydro dissection without applying tissue counter-traction. Suction may clear hydro dissection fluid and/or blood from the surgical field, and fluid irrigation may clear the suction cannula of clogging due to tissue debris and blood clots. Optionally, the instrument may include a self-contained battery-operated fluid pump and a port that allows device connection to a vacuum cannister in the operating room.

In yet another example, a hydro dissection and suction laparoscopic forceps device is provided that includes two movable jaws connected to distal end of a long rigid, e.g., five millimeter (5 mm) outer diameter, shaft, with the jaws configured to be opened and closed by an actuator on a handle on a proximal end of the shaft, e.g., including an elongated stationary ring that accommodates multiple fingers and a movable thumb ring that actuates the jaws. Tubular channels may be provided on lateral aspects of the shaft and jaws, e.g., including a pair of shaft channels extending between the proximal and distal ends of the shaft and a relatively short, e.g., three millimeter (3 mm) long, tubular jaw channel on each of the jaws, which are circumferentially exposed to allow attachment of two flexible tubes connecting each shaft channel to a corresponding jaw channel in a fluid tight fashion. The flexible tubes may allow the closed jaw instrument to maintain a desired maximum outer diameter or other profile, e.g., a five millimeter (5 mm) outer diameter throughout the length of the device, e.g., for insertion through a corresponding, e.g., five millimeter (5 mm), inner diameter trocar or sheath.

Once the jaws are disposed inside an abdominal cavity of a patient, the jaws may be opened and closed while the flexible tubes allow fluid delivery lateral to the jaws for atraumatic hydro-dissection of tissue lateral to anatomic structures grasped by the jaws, e.g., as counter traction is applied to the tissue stabilized by the forceps. Such a method of lateral tissue hydro-dissection while applying centralized tissue counter traction may be less traumatic to tissue than conventional blunt dissection with conventional laparoscopic forceps, as the such conventional forceps typically involve tissue puncture and tissue tearing as components of mechanical blunt dissection. In contrast, hydro-dissection, as enabled by the devices and methods herein, uses gentle fluid jet streams to separate tissue and isolate anatomic structures, eliminating the sharp force tissue interaction associated with mechanical blunt dissection.

In some laparoscopic procedures, it may be desirable to perform surgery with forceps containing double action jaws rather than a single action jaw and a stationary jaw. Double action jaws may permit a wider grasp of tissue to prevent slippage during tissue manipulation. Double action jaws may also enable the jaws to remain in axial orientation with the shaft of the instrument, with the jaws opening symmetrically on either side of the shaft. Application of the laparoscopic forceps with double action jaws may be more intuitive, facilitating efficient surgical technique and saving operative time. In contrast, with a single action forceps, the instrument shaft needs to be displaced to the side of the stationary jaw to accurately grasp tissue as intended. Thus, double action jaws on the device may be particularly useful, although, alternatively, single action jaws may also be provided, if desired.

In one example, the hydro-dissection laparoscopic forceps devices provided herein may incorporate a self-contained fluid pump and/or a battery or other power source to power the pump, e.g., within or on the handle device. Saline may be supplied to the pump via an intravenous line, e.g., attached to a hanging intravenous fluid bag. Optionally, a separate hose supplying wall suction in the operating room is also connected to the device handle.

In one example, the device may include two normally-closed valves coupled to respective actuators, e.g., which reside in series in the intravenous line attached to both tubular channels on the proximal end of the shaft. For example, a first trumpet valve may control both the electrical supply to the fluid pump and the fluid flow lateral to the forceps jaws, and a second trumpet valve may activate vacuum to clear fluid injected during hydro dissection.

Optionally, the laparoscopic hydro dissection forceps and/or other devices described herein, e.g., including fluid pump and battery, may be a single-use device that is disposed following the surgical procedure, to avoid the need for device cleaning, re-sterilization, and storage between successive procedures. Alternatively, all or some components of the device may be reusable, e.g., after cleaning and/or sterilization.

In one example, the fluid carrying tubular channels located on the grasper jaws may have a smaller inner diameter than the tubular channels located on the lateral aspects of the instrument shaft. This allows the velocity of the fluid jet emanating from the grasper jaw channels to be tuned to a desired level, e.g., based on pressure and/or flow rate specifications generated by the fluid pump.

Pathologic conditions may exist that cause anatomic landmarks to be obscured during endoscopic surgery, rendering tissue dissection difficult and hazardous. For example, in laparoscopic cholecystectomy or removal of the gallbladder, surgical dissection must be performed to isolate the cystic artery and cystic duct to allow for their ligation and transection prior to gallbladder removal. Acute cholecystitis is inflammation of the gallbladder caused by occlusion of the cystic duct by gallstones. The gallbladder becomes distended, and the pressure inside the organ may increase to such a level that it compromises the blood supply and causes ischemia, leading to gangrenous cholecystitis, which occurs in over 20% of acute cholecystitis cases. The severe inflammation observed in gangrenous cholecystitis causes such a degree of swelling and edema in the gallbladder and surrounding tissues that the outlines of anatomic structures, such as the cystic duct and cystic artery are invisible under laparoscopic visualization, and normally observed outlines and landmarks are obscured.

Blunt tissue dissection using conventional laparoscopic forceps requires insertion of the closed tips of the forceps jaws into tissue without perceptible landmarks, followed by opening of the jaws to spread apart the tissue. The tissue disruption associated with this blunt dissection maneuver may easily lacerate or transect unseen vessels and ducts. In contrast, hydro-dissection is a less traumatic approach to isolation of anatomic structures embedded in edematous tissue. Surgical dissection of inflamed tissue also prolongs procedure times, increasing the physical stress of surgery and general anesthesia to the patient, thus increasing the patient morbidity and mortality.

A modified technique of tissue dissection is proposed herein, involving tissue dissection performed solely by simultaneous hydro-dissection lateral to both sides of double action movable laparoscopic grasper jaws. The jaws of the devices described herein may not be applied in a typical fashion for mechanical tissue disruption and blunt dissection. Rather, the jaws may gently grasp and fixate exposed tissue prior to instillation of pressurized fluid jets lateral to the grasping jaws to perform hydro dissection of the soft tissue to achieve isolation of the desired anatomic structures.

The hydro dissection devices and methods herein may also be used in additional endoscopic procedures, e.g., to isolate delicate anatomic structures obscured by overlying amorphous tissue, such as resection of intra-abdominal endometriosis lesions, lysis of tissue and organ adhesions, and video assisted thoracic surgical procedures such as lung resection and lobectomy. In these procedures, dissection of connective tissue surrounding delicate organs, blood vessels and ducts may be performed less traumatically via hydro dissection versus standard mechanical blunt surgical dissection.

In accordance with one example, a device is provide for performing hydro-dissection of tissue within a patient's body that includes an elongate shaft comprising a proximal end, a distal end sized for introduction into the patient's body, and one or more shaft channels extending between the proximal and distal ends; first and second jaws on the distal end coupled to an actuator on the proximal end for moving the jaws between closed and opened positions, each jaw comprising a jaw channel comprising an outlet disposed adjacent a distal tip of the respective jaw; and a flexible tube extending between each jaw and the distal end of the shaft to fluidly couple the outlet of the respective jaw to the one or more shaft channels to deliver pressurized fluid from a fluid source through the one or more shaft channels, the flexible tubes, the jaw channels, and out the outlets to dissect tissue adjacent grasped between the jaws.

In accordance with another example, a device is provided for performing hydro-dissection of tissue within a patient's body that includes an elongate shaft comprising a proximal end, a distal end sized for introduction into the patient's body, and first and second shaft channels extending between the proximal and distal ends; first and second jaws on the distal end, each jaw comprising an outlet disposed adjacent a distal tip of the respective jaw; an actuator on the proximal end coupled to the jaws to manipulate the jaws between closed and opened positions; first and second flexible tubes extending between the jaws and the distal end of the shaft communicating between the outlet of the respective jaws and the first and second shaft channels, respectively; and a source of pressurized fluid coupled to the first and second shaft channels to deliver pressurized fluid through the shaft channels, the flexible tubes, the jaw channels, and out the outlets to dissect tissue adjacent grasped between the jaws.

In accordance with still another example, a method is provided for dissecting tissue within a patient's body that includes providing a dissection device comprising a distal end carrying a pair of jaws, each jaw comprising a nozzle adjacent a distal tip of the jaw; introducing the distal end into the patient's body with the jaws in a closed position; opening the jaws; manipulating the device and jaws to grasp tissue between the jaws; and delivering pressurized fluid out the nozzles to dissect tissue adjacent the jaws.

In accordance with another example, a combination hydro-dissection, irrigation, and suction laparoscopic forceps may be provided that includes a shaft, e.g., having about a three millimeter (3.0 mm) or smaller outer diameter, that resides and translates axially within a lumen of an outer sheath, e.g., a thin-walled sheath having an outer diameter of about five millimeters. The outer sheath may include a relatively large primary or central lumen and a relatively small (e.g., about 0.0325 inch (0.81 mm) inner diameter) fluid channel, e.g., incorporated in the wall the full length of the sheath, which may generate a high velocity hydro dissection jet emanating from a distal tip of the sheath. The central lumen of the sheath may be configured to supply either vacuum suction or low velocity fluid irrigation when connected to an appropriate source, as desired.

Optionally, a control valve integrated with an electrical switch may be provided that initiates fluid delivery by an attached fluid pump, with the capability to select either a high velocity jet emanating from the small diameter hydro dissection nozzle, or a low velocity fluid irrigation through the lumen of the outer sheath. Optionally, the control valve may include multiple settings, e.g., to allow different flow rates and/or volumes of fluid to be delivered, e.g., using a potentiometer and/or other control mechanism. A separate control valve produces suction through the outer sheath. In one example, an elastomeric seal maybe provided at a proximal end of the outer sheath, which may form a fluid-tight seal around the shaft of forceps while allowing the forceps to translate axially to expose the forceps jaws out of the distal end of the sheath or retract the jaws fully into the sheath.

The sheath may be constructed of a substantially rigid material, e.g., a thin-walled stainless steel tube of about 0.007 inch (0.18 mm) wall thickness, with about a 0.042 inch (1.05 mm) OD×0.0325 inch (0.81 mm) ID stainless steel tube welded or otherwise permanently axially along its inner surface to provide the fluid channel for the hydro dissection nozzle. Alternatively, the sheath may be a double lumen polymer extrusion with a small diameter, e.g., 0.0325 inch (0.81 mm), lumen incorporated in the wall of the extrusion. Exemplary materials for the extrusion may include one or more of Nylon, polyimide, polyetheretherketone (PEEK), and the like. A fluid pump and power source that powers the pump, e.g., a nine-volt battery or cable connectable to an external power source, may be attached to the body of the forceps. Alternatively, the outer sheath, fluid pump, battery, and control valves may be incorporated into a frame that accepts and rigidly attaches to a conventional laparoscopic forceps.

In tests of a hydro dissection jet with fluid delivery supplied by a twelve-volt, 400 mA diaphragm pump powered by a nine-volt battery, the water jet exhibited a velocity of 28 m/sec. This fluid velocity is sufficient to dissect connective tissue without severing or lacerating blood vessels and ducts.

In robotic surgery, the limitation of a surgeon not working next to the patient creates major issues during instrument exchange. Instruments are introduced from a dependent area and may cause organs blocking the inner opening of the port. During exchanges, there are risks that the assistant surgeon can perforate an organ as the instruments always return to their initial position during removal of the previous instrument. Any of the devices and systems herein including a camera, microfluidics, and/or other sensors may warn the introducer of organ blocking the port and potential injury.

In accordance with another example, tissue manipulation and blunt tissue dissection are conducted with the jaws of the laparoscopic forceps exposed distal to the tip of a retracted coaxial outer sheath. With the instrument in this configuration, the shaft of the laparoscopic dissector forceps occupies the central lumen of the bushing located at the distal tip of the outer sheath of the device, forcing all fluid flow to exit a small diameter nozzle in the bushing to create a high velocity fluid jet. Tissue hydro dissection is performed in this configuration, with the forceps grasping tissue to provide counter-traction while the high velocity fluid jet performs atraumatic surgical dissection without the mechanical tissue disruption typically performed by laparoscopic dissectors and graspers. Hydro dissection safely isolates and dissects ducts, blood vessels and other anatomic structures during surgical procedures in gangrenous or edematous tissue and organs.

The outer sheath of the instrument may be extended to cover the jaws of the laparoscopic dissection forceps. As the cross-sectional area of the tapered forceps jaws is less than the cross-sectional area of the forceps shaft, an enlarged fluid path is formed at the tip of the outer sheath to enable suctioning, or to provide low velocity fluid irrigation. Suction clears hydro dissection fluid and blood from the surgical field, and fluid irrigation clears the suction cannula of clogging due to tissue debris and blood clots.

In one example, the instrument has a self-contained battery-operated fluid pump and a port that allows device connection to a vacuum cannister in the operating room.

In accordance with a particular example, a hydro dissection, irrigation, and suction laparoscopic forceps is provided that includes a three millimeter (3 mm) outer diameter or smaller laparoscopic forceps that resides inside a thin walled five millimeter (5 mm) outer sheath. The outer sheath extends nearly the full length of the shaft of the forceps, and is configured to translate axially along the forceps shaft to either fully expose or fully enclose the forceps jaws. Axial translation of the outer sheath is performed via an actuator located on the proximal handle of the device.

A bushing is attached to the distal tip of the outer sheath. In one example, the bushing is approximately twelve millimeters (12 mm) long, and includes a lumen approximately four millimeters (4 mm) in inner diameter therethrough. A one millimeter (1 mm) thick end cap on the bushing includes a central lumen with an inner diameter that is a sliding fit with the outer diameter of the laparoscopic forceps, and a tiny offset lumen approximately 0.3 millimeter in diameter.

When the outer sheath is fully retracted, the central lumen of the end cap on the bushing seals against the outer surface of the dissector forceps shaft, and all fluid flow exits the 0.3 mm diameter lumen, forming a high velocity fluid jet. When the outer sheath is fully extended to enclose the forceps jaws, injected fluid flows through a low resistance path, with ample clearance between the dissector forceps shaft and the inner surface of the outer sheath, and between the outer surface of the forceps jaws and the central lumen of the bushing end cap, to produce low velocity fluid irrigation, or to allow vacuum suction to occur.

In one example, fluid flow is provided via a battery powered fluid pump integrated into the device handle, with a fluid line connecting the pump to the fluid supply consisting of an elevated intravenous saline bag. A separate connector in the handle attaches to a vacuum line that utilizes a standard operating room vacuum source. A specialized spring-loaded three-way control valve is included on the device handle, which may be actuated between two positions, to provide either fluid flow or vacuum to the lumen of the outer sheath. In its normal resting position, the valve permits fluid flow to occur. When the suction control button is pressed, the valve shuttles to its second position, cutting off fluid flow while opening the vacuum line.

In one example, fluid flow is provided by a battery powered electric pump activated by depression of an electrical button switch in the device handle. Two nine-volt batteries supply power to the pump via an electronic control board that maintains a constant voltage delivery to ensure that a constant fluid jet velocity is achieved for reliable tissue hydro dissection. A rotational switch that controls a potentiometer may also be provided on the instrument handle to allow the surgeon to adjust the velocity of the hydro dissection jet.

Other aspects and features of the present invention will become apparent from consideration of the following description taken in conjunction with the accompanying drawings.

### BRIEF DESCRIPTION OF THE DRAWINGS

The invention is best understood from the following detailed description when read in conjunction with the accompanying drawings. It is emphasized that, according to common practice, the various features and design elements of the drawings are not to-scale. On the contrary, the dimensions of the various features and design elements are arbitrarily expanded or reduced for clarity. Included in the drawings are the following figures.

FIGS. 1a and 1b depict the appearance of an exemplary non-gangrenous and gangrenous gallbladder, respectively.

FIGS. 2a and 2b show exemplary maneuvers used in mechanical blunt dissection of tissue.

FIGS. 3a-3c depict an exemplary sequence of steps that may be used in hydro dissection of tissue, e.g., using the hydro-dissection devices described herein.

FIG. 4 illustrates exemplary differences in width of jaw opening between a single action jaw instrument and a double action jaw instrument.

FIG. 5 depicts exemplary components of a hydro dissection laparoscopic forceps device.

FIGS. 6a-6c illustrate details of a distal portion of the device of FIG. 5.

FIGS. 7a and 7b depict an exemplary profile of the jaws of the device of FIG. 5 in closed and open configurations, respectively.

FIG. 8a shows another example of an apparatus that may provide hydro-dissection, irrigation and suction in a unitary format including a forceps movable relative to an outer sheath.

FIG. 8b is a cross-sectional view of the device of FIG. 8a taken along section 8b-8b.

FIG. 8c is a cross-sectional view of an alternative configuration of the device of FIG. 8a.

FIG. 9a shows the device of FIG. 8a with the forceps advanced relative to the sheath to allow mechanical dissection, irrigation, and/or hydro-dissection.

FIG. 9b shows the device of FIG. 8a with the forceps retracted into the sheath to allow hydro-dissection, irrigation, and/or suction.

FIG. 10 is a schematic showing exemplary fluid pathways (solid lines) and electrical pathways (dashed) that may be included in the device of FIG. 8a.

FIG. 11a shows another example of an apparatus including a framework including an outer sheath for receiving a separate forceps instrument.

FIG. 11b shows the apparatus of FIG. 11a with the forceps advanced such that jaws extend from a distal end of the sheath.

FIG. 11c is a bottom view of the apparatus of FIGS. 11a and 11b.

FIG. 12 is a table showing results of exemplary water jet flow rate testing.

FIG. 13a shows another example of an apparatus that provide hydro-dissection, irrigation and suction in a unitary format including a forceps movable relative to an outer sheath connected to a robotic control system.

FIG. 13b shows an exemplary forceps apparatus connectable to a robotic arm of a robotic surgical system.

FIGS. 14a-14f show the apparatus of FIG. 13 with the forceps deployed from (FIGS. 14a-14c) and retracted into (FIGS. 14d-14f) the sheath and selectively used to deliver a hydro-dissection jet or irrigation.

FIGS. 15a-15f are cross-sectional details of a distal end of the apparatus as shown in FIGS. 14a-14f, respectively.

FIG. 16 illustrates the layout of the components included in an exemplary hydro dissection, irrigation, and suction laparoscopic forceps device.

FIGS. 17a-17e illustrate activation of a low velocity fluid irrigation and a high velocity fluid jet with the hydro dissection, irrigation, and suction laparoscopic forceps.

FIG. 18a-18b show control button activation of fluid flow and suction modes for the device.

FIGS. 19a-19b depicts the two configurations of the specialized valve that selects for either fluid flow or suction via the distal tip of the device.

FIGS. 20a-20d illustrates the difference in effective vacuum flow area between the current device and the previous device containing a full-length fluid jet channel.

FIG. 21 illustrates an example of a hydro dissection, irrigation and suction laparoscopic forceps instrument that includes controlled electrocautery capability.

FIGS. 22a and 22b are side views of the instrument with a coaxial sheath in advanced and retracted positions, respectively, to cover and expose jaws of a forceps disposed within the sheath.

FIGS. 22c and 22d are details of a distal end of the instrument of FIGS. 22a and 22b in the advanced and retracted positions, respectively, showing low velocity fluid irrigation and in FIG. 22c and high velocity fluid delivery for tissue dissection in FIG. 22d, where the jaws may also be used to provide electrocautery capability.

FIGS. 22e and 22f are details of the instrument of FIG. 21 showing an electrical switch mechanism that allows electrocautery current to be delivered when the instrument is in the retracted position shown in FIGS. 22b and 22d.

### DETAILED DESCRIPTION

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

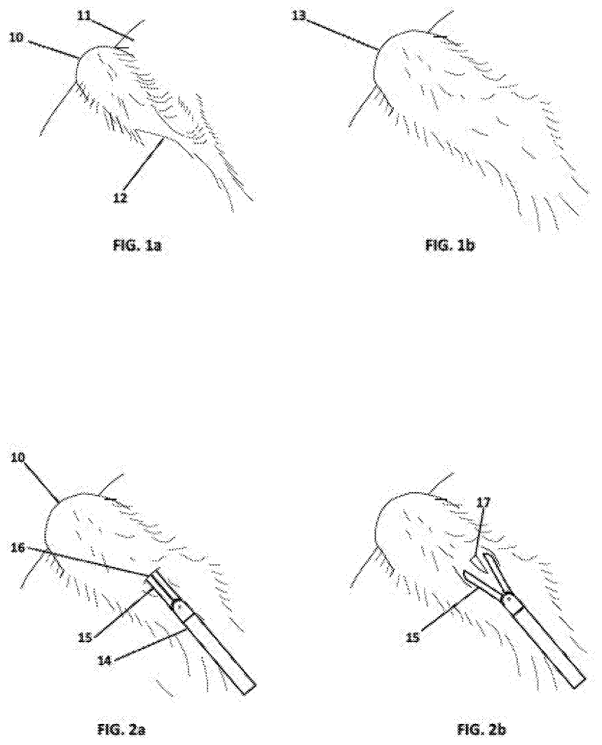

Turning to the drawings, FIG. 1a illustrates an example of the surgical appearance of a gallbladder 10 attached to the underside of a liver 11, in a non-gangrenous situation. The outline of the cystic duct 12 may typically be observed in a non-gangrenous laparoscopic cholecystectomy surgery. FIG. 1b depicts an example of a gangrenous gallbladder 13, that is enlarged, swollen and edematous. The outline of the gangrenous gallbladder 13 and normal surrounding anatomical landmarks are obscured by the inflamed and edematous tissue.

FIG. 2a depicts an example of mechanical blunt dissection of the gallbladder 10 using conventional laparoscopic forceps 14. The tips of the jaws 15 of the laparoscopic forceps 14 are inserted into tissue at an entry site 16, with the jaws 15 in a closed orientation. Following tissue insertion, the jaws 15 are forcibly opened, as shown in FIG. 2b, to dissect the tissue of gallbladder 10, creating a cleavage plane or opening 17 in the bluntly dissected tissue. Blunt mechanical dissection of tissue is generally safe to perform when the gallbladder is non-gangrenous, and underlying anatomical structures such as blood vessels and ducts are visually perceived. If underlying blood vessels and ducts are obscured, however, e.g., due to tissue edema and inflammation in the event of gangrenous cholecystitis and the like, laceration, perforation, and/or transection of blood vessels and ducts may occur upon blunt dissection maneuvers using conventional laparoscopic forceps. Application of a fluid jet to dissect tissue in gangrenous gallbladder may be useful, as a moderately pressurized fluid jet imparts less force than rigid forceps jaws during tissue dissection, particularly in inflamed and edematous tissue that is more friable than normal tissue.

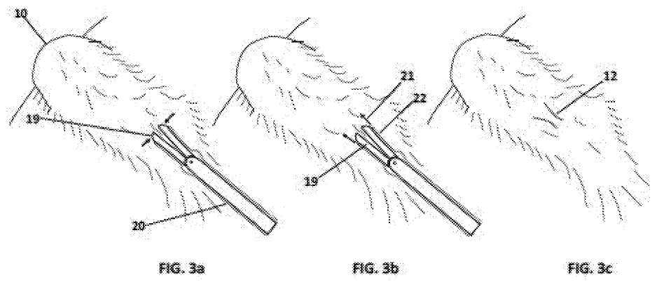

FIG. 3a-3c illustrate an exemplary technique of atraumatic tissue dissection using a hydro dissection laparoscopic forceps device 20. In FIG. 3a, the jaws 19 of the hydro dissection forceps 20 gently close on tissue of the gallbladder 10 without its tips puncturing into the tissue. Following closure of the jaws 19, the hydro dissection forceps device 20 may remain in a fixed position, i.e., the device 20 is not moved to tear or otherwise displace tissue. In FIG. 3b, following fixation of the tissue by closure of the jaws 19, fluid jets 21 are initiated, e.g., lateral to each jaw 19. The closed jaws 19 fixate tissue and provide counter traction as the fluid jets 21 exert hydro dissection force against tissue lateral to the region grasped by the jaws 19. Tissue separation is performed solely by hydro dissection, and avoidance of conventional blunt dissection maneuvers involving tissue tearing and tissue puncture leads to atraumatic isolation of anatomic structures, such as the duct 12 observed in FIG. 3c.

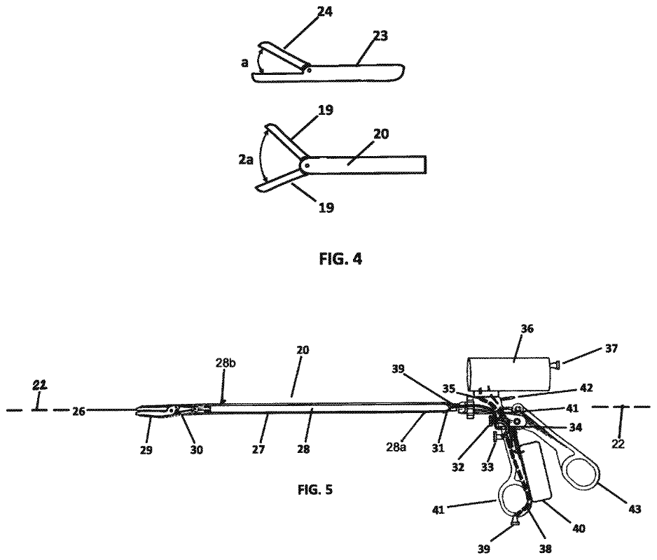

FIG. 4 illustrates functional differences that may be experienced between forceps 23 with a single action jaw 24 and double action forceps 20 with two moveable jaws 19. Jaws 19 of double action forceps 20 open twice as wide as forceps 23 with a single moveable jaw 24, permitting a surgeon to grasp a larger amount of tissue between the jaws 19 for enhanced tissue control and avoidance of tissue slippage out of the grasp of jaws 19. Proper tissue fixation is necessary to provide requisite counter traction during lateral hydro dissection procedures, particularly with the wet tissue environment encountered with the technique. Thus, double action, movable jaws 19 may provide advantages over a single action jaw 24 during tissue dissection.

Turning to FIG. 5, exemplary components are shown that may be included in a hydro-dissection laparoscopic graspers device 20. Generally, the device 20 includes an elongate shaft 28 defining a longitudinal axis 22, e.g., a substantially rigid tubular or solid shaft, including a proximal end 28a and a distal end 28b sized for introduction into a patient's body. The shaft 28 may include a pair of shaft channels 27, e.g., including diametrically opposed rigid fluid supply tubes 27 attached to lateral aspects of the shaft 28. Alternatively, the shaft channels 27 may be integrally formed in the wall of the shaft 28, e.g., by one or more of extrusion, molding, casting, machining, and the like. In a further alternative, the shaft 28 may include a single channel or infusion lumen extending from the proximal end 28a to the distal end 28b and a fitting split, or other branch (not shown) may be provided on the distal end to provide two openings that may be connected to respective flexible tubes and jaw channels 29.

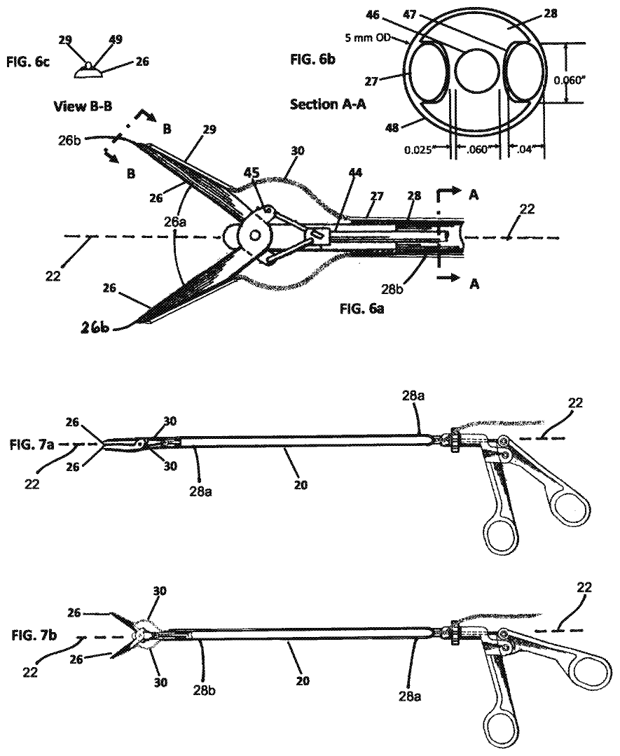

The device 20 includes a pair of jaws 26 on the distal end 28b of the shaft 28, e.g., that may be manipulated between a closed position (e.g., as shown in FIG. 7a) and an open position (e.g., as shown in FIG. 7b), via manipulation of an actuator 43 on a handle 41 on the proximal end 28a of the shaft 28. In the example shown, the handle 41 includes a stationary ring extension 41 and a moveable ring actuator 43 coupled to the jaws 26 to direct them between the closed and open positions. As best seen in FIG. 6a, the jaws 26 are coupled to the distal end 28b of the shaft 28 such that, manipulation of the actuator 43 causes both jaws 26 to move laterally away from the axis 22 when the jaws 26 are opened and the jaws 26 are generally aligned along the axis 22 when the jaws are closed, e.g., as shown in FIGS. 5 and 7a. Alternatively, the device may include only a single movable jaw and a fixed jaw attached to or otherwise extending from the distal end of the shaft (not shown), e.g., similar to the forceps 23 shown in FIG. 4. In this alternative, an actuator may move the single jaw between open and closed positions to grasp tissue between the jaws.

Optionally, the jaws 26 may include substantially blunt surfaces to prevent puncturing, cutting, and/or otherwise damaging tissue. For example, as shown, the inner contact surfaces 26a and/or the distal tips 26b of the jaws 26 may include flat and/or rounded edges to allow tissue to be grasped between the jaws 26 with minimal risk of tearing or cutting. Alternatively, a different end effector may be provided than the blunt jaws 26. For example, the jaws may include sharpened inner edges and/or pointed tips, e.g., to provide a scissors or other cutting instrument (not shown). In another alternative, a stapler or clip applier may be provided for the end effector, e.g., including one or more staples or clips carried by one of the jaws and an anvil or other structure on the opposite jaw (not shown) to allow one or more staplers or clips to be applied through tissue contacted between the jaws.

As best seen in FIG. 6a, fluid supply tubes 29 may be attached and/or otherwise provided on the jaws 26, e.g., extending along lateral aspects of the jaws 26 on opposite outer longitudinal edges. For example, separate substantially rigid tubular segments may be formed and permanently attached to the outer edges of the jaws 26, e.g., by one or more of bonding with adhesive, welding, soldering, brazing, fusing, and the like, or, alternatively, the jaw tubes 29 may be integrally formed with the jaws 26. A flexible tube 30 extends from each shaft channel 27 to a respective jaw tube 29, e.g., attaching the distal end of shaft fluid supply tubes 27 to jaw fluid supply tubes 29 in a fluid tight manner. The flexible tubes 30 allow fluid delivery while the jaws 26 are opened, closed, or partially open.

In one example, when the jaws 26 are closed, the outer profile of the working portion of the device 20, including the jaws 26, fluid supply channels 29 and 27, and flexible tubes 30 do not exceed about five millimeters (5 mm) or other maximum outer diameter or cross-section, e.g., to allow the device 20 to be introduced into the body through a corresponding sized access device, e.g., a five millimeter (5 mm) laparoscopic or thoracoscopic trocar, delivery sheath, and the like (not shown).

Optionally, the graspers 20 may be connected to a radiofrequency power source (not shown), e.g., via a connector 42 on the proximal end 28a, e.g., on handle 41 as shown in FIG. 5, to allow the jaws 26 to cauterize blood vessels, ducts, and/or other tissue, similar to conventional laparoscopic graspers. The connector 42 may be coupled to the jaws 26 by one or wires or other leads (not shown) extending between the proximal and distal ends 28a, 28b of the shaft 28, which may, in turn, to electrically conductive electrodes or surfaces on the jaws 26. For example, the entire inner contact surface 26a of the jaws may be coupled to the leads to deliver electrical energy to tissue contacted between the jaws 26, if desired. Optionally, the jaws 26 may include one or more sensors, e.g., a Doppler sensor (not shown) on one of the jaws that may be used to identify blood flow in tissue captured between the jaws or otherwise contacted by the sensor(s). A processor (not shown) may be coupled to the sensor(s) and/or cautery elements to provide an output to the user when blood is flowing and/or discontinued, e.g., for use in conjunction with cauterizing contacted tissue.

Pressurized fluid for hydro-dissection is supplied by a miniature fluid pump 36 included in or coupled to the handle 41, e.g., integrated into the superior aspect of the stationary portion of the handle 41. The fluid pump 36 may include a connector, e.g., a female luer fitting 37, that accepts an intravenous fluid line connected to a saline bag or other source of fluid (not shown), e.g., containing one to three (1-3) liters or other desired volume of sterile saline. In one example, a fluid supply line 35 extends from the pump 36 to a normally closed fluid irrigation trumpet valve 32 provided on the handle 41.

Optionally, a connector 39 may be provided to connect a source of vacuum or suction (not shown) to the device 20, e.g., communicating with the shaft channels 27. For example, as shown in FIG. 5, the stationary handle portion 41 may include a suction luer fitting 39 that allows the hydro-dissection laparoscopic graspers 20 to be connected to wall suction in the operating or procedure room, for evacuation of fluid via jaw channels 29. As shown, a suction connecting tube 38 extends from luer fitting 39 to the inlet of a suction trumpet valve 33, which is also in a normally closed position until the trumpet valve 33 is depressed.

In one example, the outlets of both irrigation trumpet valve 32 and suction trumpet valve 33 are connected together to a common fitting 34 with a connecting line 39 extending to a connector 31 that is attached to both fluid supply tubes 27. Thus, in this example, depression of the irrigation trumpet valve 32 causes fluid dissection jets to emanate from both fluid supply tubes 27, while depression of the suction trumpet valve 33 causes suction of fluid via the shaft and jaw channels 27, 29. If the jaw and/or shaft channels 29, 27 become clogged during actuation in the suction mode, the irrigation mode may be activated to clear debris lodged in channels 29, 27.

FIG. 6a depicts an enlarged view of the jaws 26 and the distal end 28b of the shaft 28 of the device 20. Each jaw channel 29 is attached to or otherwise extends from the lateral aspect of jaw 26, e.g., along the outer edge of each jaw 29. A proximal end of each jaw channel 29 may be offset distally from the hinge connecting the jaws 29, e.g. offset about three millimeters (3 mm) from the proximal end of each jaw 26, e.g., to provide circumferential clearance to permit attachment of the flexible tube 30 and/or accommodate rotational movement of the jaws 26. Similarly, each shaft channel 27 is attached to or otherwise extends axially along either side of the shaft 28, except for desired offset, e.g., about a three millimeters (3 mm) long length of its distal portion, where circumferential clearance permits attachment of the proximal end of flexible tube 30.

An actuator may be coupled to the jaws 26 to manipulate the jaws between the open and closed positions. For example, as shown, the jaws 26 are opened and closed via advancement and retraction using an elongate member, e.g., a stainless steel rod 44 that extends through the length of shaft 28 and connects to jaw actuation linkage 45. FIG. 6b is a cross-section of the shaft 28, showing a central channel 46 that accommodates actuation rod 44 in a sliding fashion within its lumen. In the example shown, two lateral grooves 47 may extend the length of shaft 28, to allow the attachment of fluid tubes 27, which may be elliptical rather than circular in cross-section, if desired, to increase its luminal area for maximal fluid delivery. Optionally, a relatively thin outer sheath 48, e.g., composed of polymer heat shrink or other material, may cover the shaft 28 and attached fluid tubes 27, to provide a substantially smooth outer surface for insertion and sealing in a laparoscopic trocar port, and/or to electrically insulate the outside of shaft 28 as radiofrequency current is applied to cauterize tissue grasped by jaws 26.

FIG. 6c is an end view of an exemplary instrument jaw 26, illustrating the attachment of a fluid tube 29 to its lateral aspect. If desired, the jaw 26 may be thinned out, or a groove may be cut along its length to allow attachment of fluid tube 29 while maintaining a desired outer profile, e.g., not more than about five millimeters (5 mm) when jaws 26 are closed. The fluid tube 29 may be attached to the lateral aspect of jaw 26 using one or more of adhesive, solder, braze, weld, and the like. The attachment material may form a contoured fillet 49 along the length of the attached tube 29, to yield a smooth profile of the lateral aspect of the jaw 26, for atraumatic tissue contact during surgery.

FIG. 7a depicts the hydro-dissection laparoscopic forceps 20 with the jaws 26 in a closed position. In the example shown, the flexible fluid supply connecting tubes 30 remain substantially flush against the forceps 20 when jaws 26 are closed, allowing unimpeded insertion through a desired access device, e.g., a 5 mm endoscopic trocar (not shown). FIG. 7b shows forceps 20 with the jaws 26 in an open position. Flexible connecting tubes 30 splay out laterally when the jaws 26 are opened. Forceps 20 insertion and removal must be conducted only with closed instrument jaws 26.

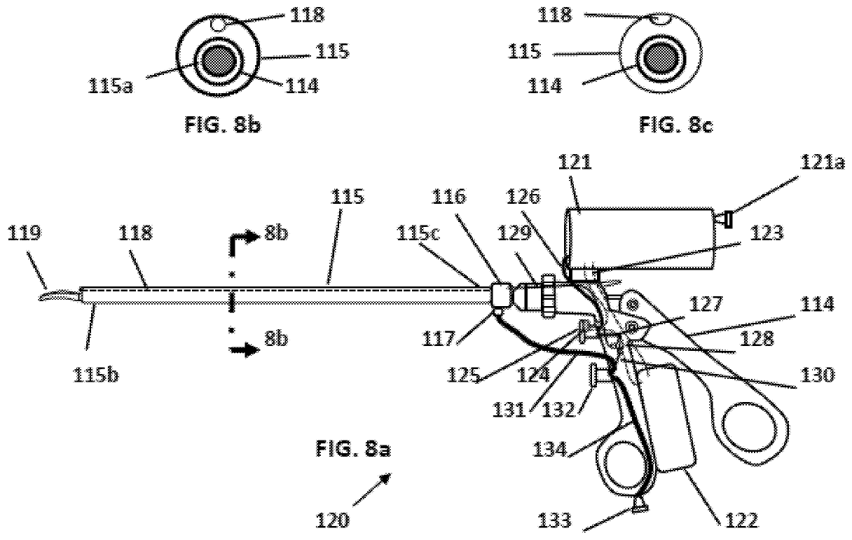

Turning to FIG. 8a, another example of an apparatus 120 is shown that includes an outer sheath 115 and a forceps instrument 114 that may be used to selectively provide hydro-dissection, irrigation, and/or suction, e.g., as needed during a surgical procedure. In the example shown, the forceps 114 and outer sheath 115 may be manufactured and provided separately but, before or during a procedure, the forceps 114 may be inserted into the sheath 115 to allow the different modes of operation. For example, the sheath 115 may be made to accommodate inserting a conventional forceps instrument (or alternatively, another instrument, such as a bowel grasper, scissors, clip applier, vessel sealer, and the like not shown) to provide hydro-dissection and/or irrigation/suction during use of the conventional instrument. Alternatively, the forceps 114 may be manufactured integrally with the sheath 115, yet may be movable axially to advance or retract the forceps 114 as desired during a procedure.

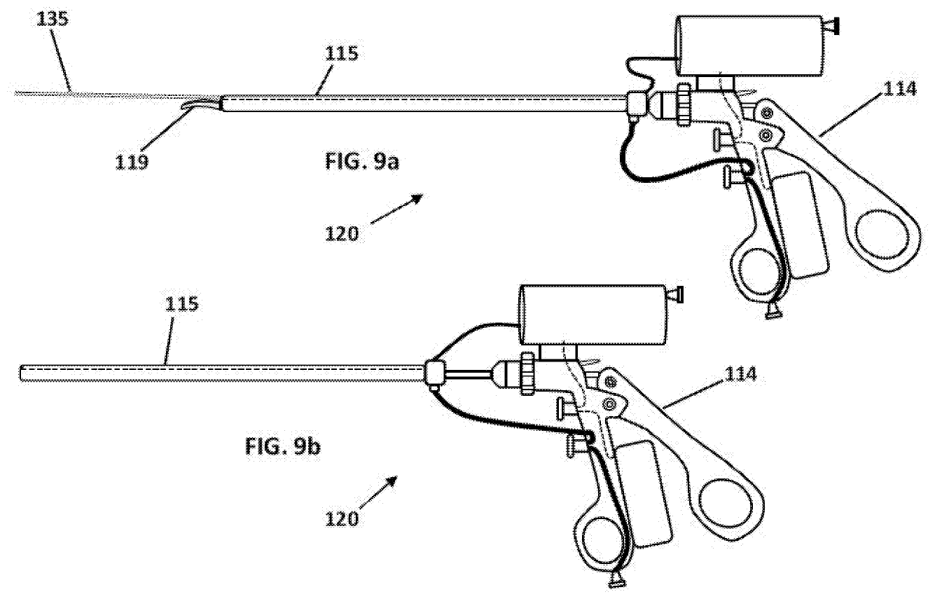

For example, as shown, a conventional three millimeter (3 mm) or smaller laparoscopic forceps 114 may be provided that is inserted through a primary or central lumen 115a of the outer sheath 115 (e.g., shown in FIG. 8b), e.g., such that jaws 119 of the forceps 114 extend distal to a distal tip 115b of sheath 115, e.g., as shown in FIGS. 8a and 9a. A fluid tight valve or other seal 116 may be provided on a proximal end 115c of sheath 115, e.g., such that the valve 116 slidably seals against the shaft of the forceps 114, and allows axial and/or rotational movement of the forceps 114 and sheath 115 relative to one another. For example, the valve 116 may allow advancement of the sheath 115 to retract the jaws 119 of the forceps 114 into the lumen 115a of the sheath 115, e.g., as shown in FIG. 9b, as well as rotation, while providing a fluid-tight seal to prevent fluid introduced into the lumen 115a from leaking.

The sheath 115 may include one or more ports communicating with the central lumen 115a, e.g., to allow fluid and/or suction to be applied. For example, as shown, a side port 117 is provided on the valve 116, which may be coupled to a source of fluid or vacuum to allow injection or removal of fluid in the surgical field via the sheath 115, i.e., through the distal opening of the primary lumen 115a at the tip 115b.

In addition, the sheath 115 may include one or more additional or secondary lumens or channels extending between the proximal and distal ends 115c, 115b. For example, as shown, a relatively small, e.g., about 0.0325 inch (0.81 mm) inner diameter, hydro dissection fluid channel 18 may be attached to the sheath 115, e.g., extending along an inner surface of the sheath 115 adjacent the central lumen 115a, as shown in FIG. 8b. The secondary fluid channel 118 may extend the length of the sheath 115 to provide an outlet at the distal tip 115b, which may be used to generate a high velocity jet for tissue dissection, as explained further elsewhere herein. Thus, relatively low velocity fluid irrigation may be supplied via the central lumen 115a of the sheath 115, and/or high velocity jet may be supplied via the secondary channel 118, as selected at any given time by a surgeon or other use.

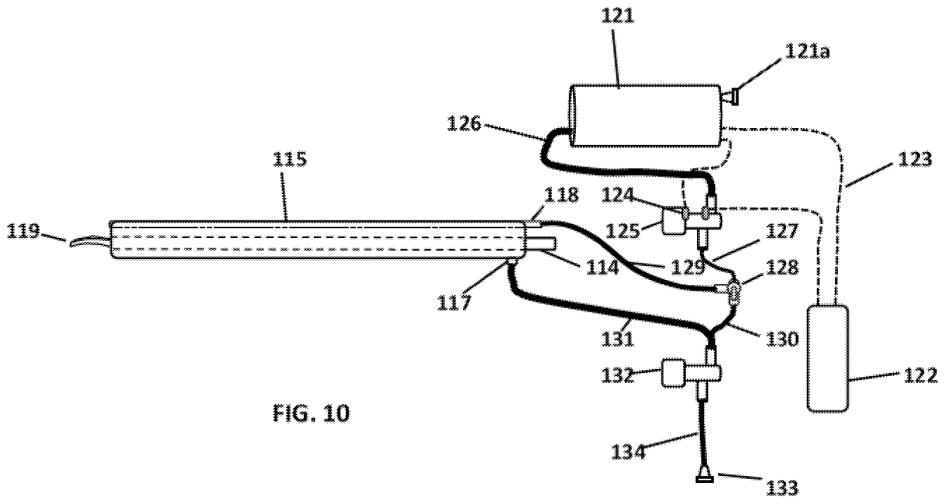

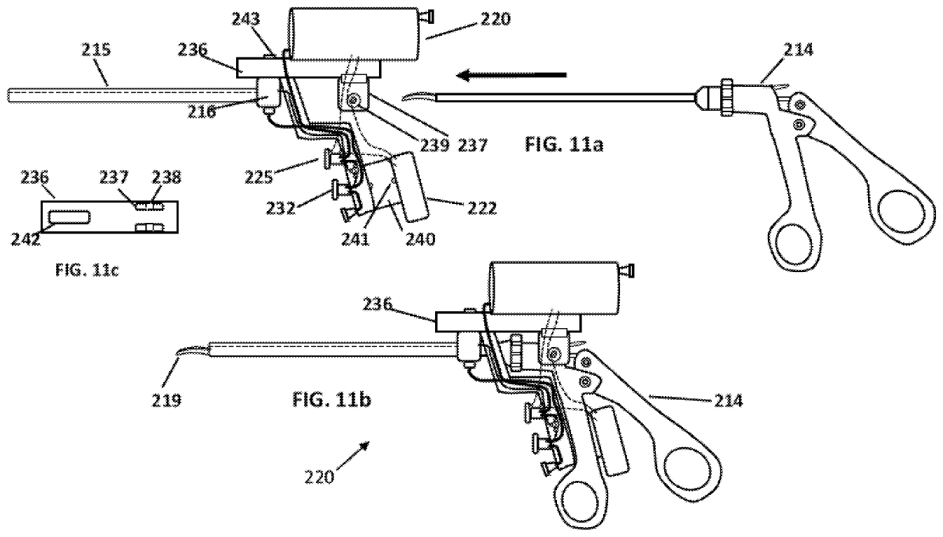

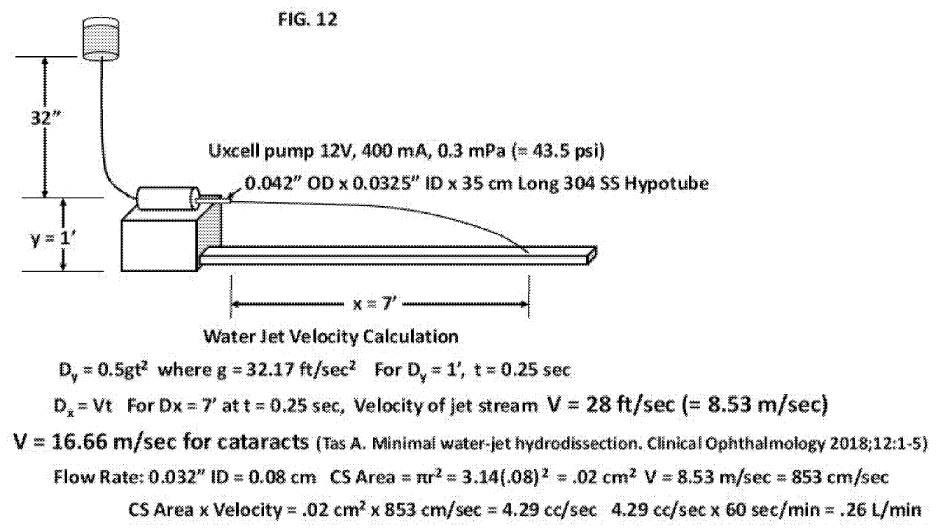

In the example shown, the fluid flow may be driven by a battery powered diaphragm pump or other fluid source 121 mounted or otherwise provided on the apparatus 120, e.g., rigidly attached to a superior aspect of a handle of the forceps 114. Alternatively, an external pump or other fluid source (not shown) may be provided that may be connected to the apparatus 120. A luer fitting or other connector 121a may be provided on the pump 121 that may be connected to a source of fluid, e.g., a line from an intravenous saline bag (not shown). Pressurized fluid may exit the pump 121 via pump supply line 126, which connects to a fluid control valve 125. In one example, the fluid control valve 125 may be a trumpet valve that includes an electrical switch 124, e.g., leading from a nine-volt battery or other power source 122 to the pump 20, e.g., as best seen in FIG. 10. Depression of the fluid control valve 125 opens the valve 125 to fluid flow and may power the pump 120 simultaneously.

In the exemplary schematic shown in FIG. 10, the output from the fluid control valve a25 leads to a three-way stopcock a28 or other control, that allows the user to select either hydro-dissection flow line 129 to the channel 118, or low velocity irrigation via the central lumen 115a of the sheath 115. Alternatively, the device may include a processor or controller (not shown) that may automatically open and close the appropriate flow line based on the actuator pressed by the operator to alternatively deliver hydro-dissection or irrigation flow. As shown, a low velocity irrigation line 130 exits the stopcock 128, and taps into an output suction line 117 exiting suction control valve 132. The input line 134 to a suction control valve 132 leads to a vacuum fitting 133, e.g., on the inferior aspect of the handle of the forceps 114. The fitting 133 may be connected to a source of vacuum, e.g., a line from the operating room vacuum source to provide suction capability via the central lumen 115a of the sheath 115, e.g., when the suction control valve 132 is depressed. Alternatively, low velocity fluid irrigation may be delivered through the central lumen 115 of the sheath 115, e.g., to unclog the apparatus 120 when tissue debris and blood clots have reduced the suction capability.

FIG. 8b is an exemplary cross-sectional view of the sheath 115, illustrating a small diameter tubular body attached to a superior aspect of the central lumen 115a of the sheath 115 to provide the secondary channel 118. As shown, the shaft of the forceps 114 occupies a portion of the central lumen 115a while providing area around the shaft to accommodate irrigation and/or suction. FIG. 8C is a cross-sectional view of an alternative construction of the sheath 115, in which the sheath 115 is a polymer extrusion or other integral tubular body that includes a secondary lumen 118 integrally formed within the wall of the sheath 115 adjacent the primary lumen 115a.

FIG. 10 shows an exemplary schematic depicting the fluid and electrical systems of the apparatus 120. In the example shown, the fluid diaphragm pump 121 receives fluid input via luer fitting 121a, and fluid exits via the output line 126. The battery 122 supplies power to the pump 121 via conducting electrodes 123, with a power switch 124 integrated into the fluid control trumpet valve 125. The fluid control trumpet valve 125 is normally in an off position, and power switch 124 normally in an open position. When the fluid control trumpet valve 125 is depressed, the valve 125 opens to fluid flow, and the power switch 124 is simultaneously closed to supply current to the pump 20. The fluid output line 127 from the fluid control valve 125 forms the input into the three-way stopcock 128, which allows the surgeon to select one of two outputs-either hydro dissection output line 129 connecting to the small diameter fluid supply channel 118, or the low velocity irrigation output line 130. The irrigation output line 130 connects to suction supply line 131, which leads from the output of suction control valve 132 to the side port 117 in communication with the central lumen 115a of the sheath 115. Suction is provided by connection of the operating room vacuum source to suction connector 133, with the suction line 134 forming the input to the suction control valve 132. When low velocity irrigation is selected via depression of fluid control valve 125 with the three-way stopcock 128 set to the irrigation output line 130, the suction control valve 132 is closed, and fluid flows through the suction supply line 131 into the central lumen 115a of the sheath 115.

FIG. 9a depicts the apparatus 120 in the configuration with the sheath 115 retracted (or the forceps 114 advanced) to expose the jaws 119 of the forceps. In this configuration, the jaws 119 may be used to perform one or both of the following techniques: (1) surgical tissue manipulation and mechanical blunt dissection; and (2) hydro dissection with tissue counter-traction, i.e., by first grasping tissue between the jaws 119 before activating the jet. As described elsewhere herein, hydro dissection with tissue counter-traction may be performed by holding tissue stationary with the forceps jaws 119 while high velocity fluid jet 135 emanates from the distal tip 115b of the sheath 115 to gently dissect tissue, avoiding tissue, blood vessel and duct disruption associated with mechanical blunt dissection. FIG. 9b depicts the apparatus 120 after retraction of the forceps 114 such that the jaws 119 are withdrawn completely into the sheath 115. This configuration may be used to perform one or more of the following techniques: (1) pure hydro dissection without tissue counter-traction; (2) suction via the central lumen 115a of the sheath 115, e.g., to remove fluid and debris from the surgical field; and (3) low velocity fluid irrigation, e.g., to remove blood from the surgical field and/or to clear the sheath 115 of blood clots or tissue debris that clog its central lumen.

Turning to FIGS. 11a and 11b, another example of an apparatus 220 is shown that may selectively provide hydro dissection, irrigation and/or suction using a laparoscopic forceps formed of two discrete devices-a frame 236 including an outer sheath 215, fluid pump 220, battery or power source 222, and control valves 225, 232 mounted on the frame 236; and a laparoscopic forceps 214 (or other instrument), generally similar to the previous examples. The laparoscopic forceps 214 may be inserted into the sheath 215, and secured rigidly to the frame 236, e.g., into a channel 237 in the frame 236 using one or more connectors, e.g., setscrews 239 that secure the frame 236 to the handle of the forceps 214. In addition or alternatively, the stationary handle portion of the forceps 214 may be secured to the frame, e.g., snapped into a grooved section 240 of the frame 236 that includes one or more detents 241 in its walls, e.g., to further secure laparoscopic forceps 214.

The sheath 215 may be movable axially relative to the frame 236, e.g., to extend and retract with respect to frame 236 using a pin or other actuator 243, e.g., attached to the valve body 216 of the sheath 215, that translates within a slot 242 in the frame 236, as shown in FIG. 11c. Alternatively, the forceps 214 may be movable axially relative to the sheath 215 between distal and proximal positions. FIG. 11b shows the assembled configuration of the forceps 214 in the hydro dissection frame 236, with the forceps jaws 219 in the distal position, i.e., extended distal to the tip of the sheath 215. FIG. 11c shows the underside of the frame 236, with the sides of the channel 237 containing threaded holes 238 that accept setscrews for attachment to the handle of the forceps 214. The slot 242 that forms part of the translation mechanism for sheath 215 is also visible.

Thus, during use, tissue may be grasped by the jaws 219, and a hydro-dissection fluid jet may be generated to dissect tissue. If desired, the jaws 219 may be retracted into the sheath 215 and a hydro-dissection fluid jet may be delivered without traction and/or irrigation/suction may be generated. Optionally, the forceps 214 may be rotatable relative to the sheath 215, e.g., to adjust the orientation of the jaws 218 when extended to facilitate grasping tissue. Optionally, the forceps 214 may be removable entirely from the sheath 215 while the distal end 215b of the sheath 215 is positioned within a surgical space, e.g., to allow one or more different instruments to be introduced through the sheath 215 to perform additional steps of the surgical procedure.

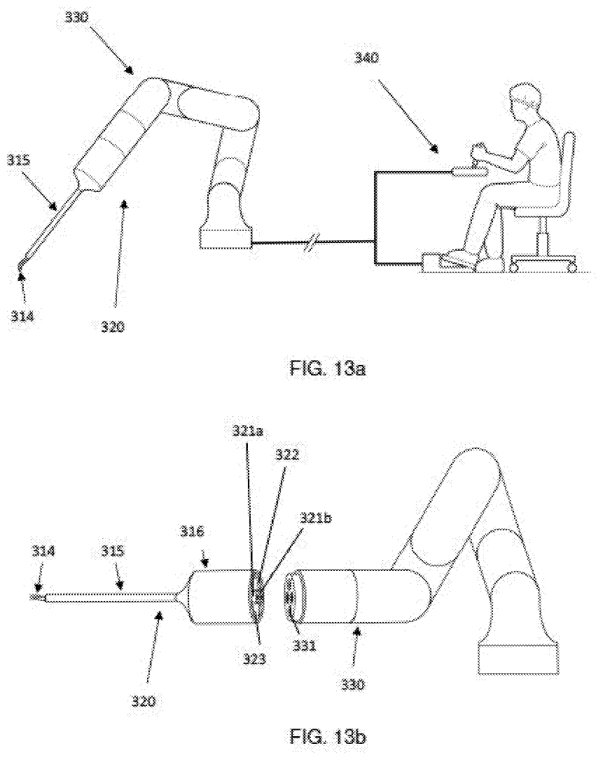

Turning to FIGS. 13-15, another example of a surgical apparatus 320 is shown that includes an outer sheath 315 and a forceps or other instrument 314 including an end effector, e.g., jaws 319, deployable from the sheath 315. Unlike the previous apparatus, the sheath 315 and forceps 314 are mounted to a robotic control arm or system 330, which is coupled to an operator console or system 340, which may be operated remotely by a surgeon. For example, as best seen in FIG. 13B, the sheath 315 may include a proximal mount or housing 316 on its proximal end that may include one or more connectors (not shown) for mounting to an end of the robotic arm system 330. For example, the housing 316 and robotic arm may include one or more mating threads, detents, latches, and the like (not shown) that fix the sheath 315 relative to the robotic arm system 330 such that, once attached, the sheath 315 is manipulated by actuating the robotic arm system 330. As shown, the housing 316 may include one or more ports, knobs, nipples, or other connectors 321-323, for connecting elements of the apparatus 320 to corresponding elements 331 on the robotic arm system 330, as described further elsewhere herein.

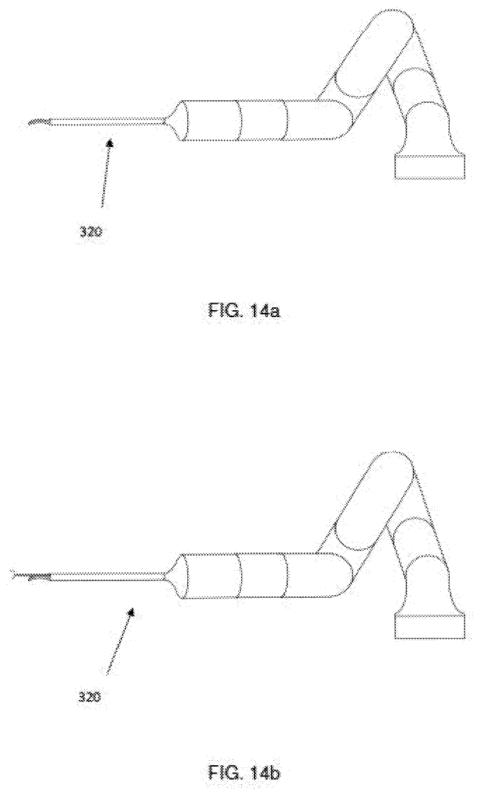

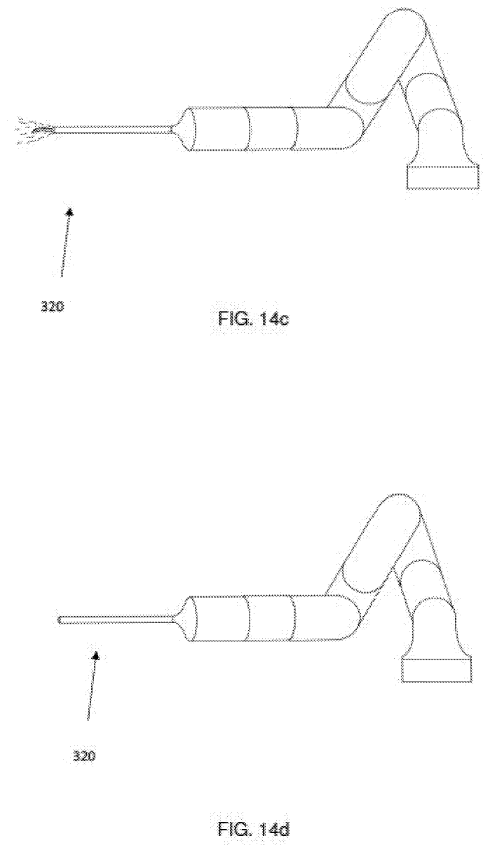

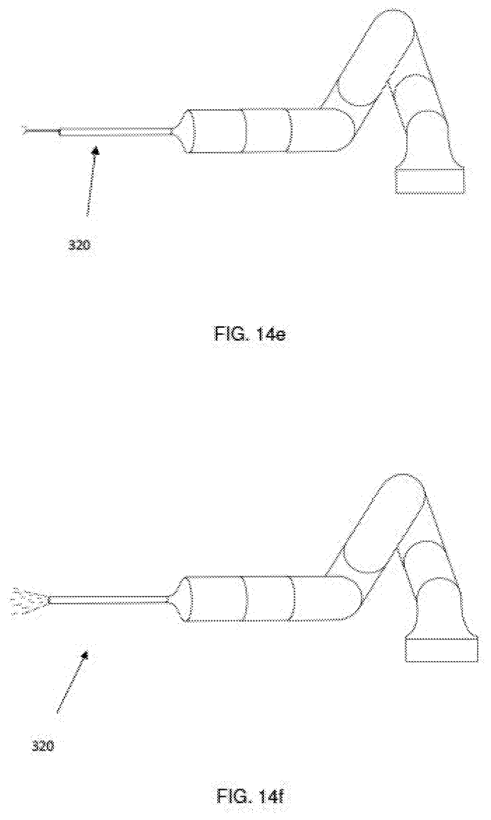

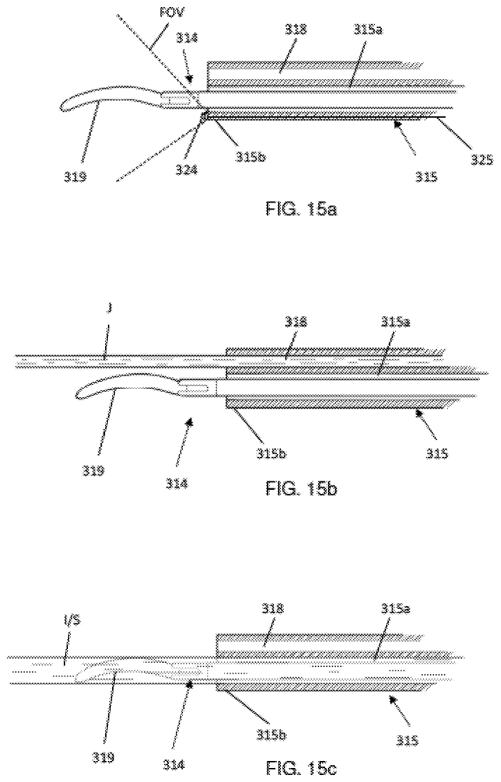

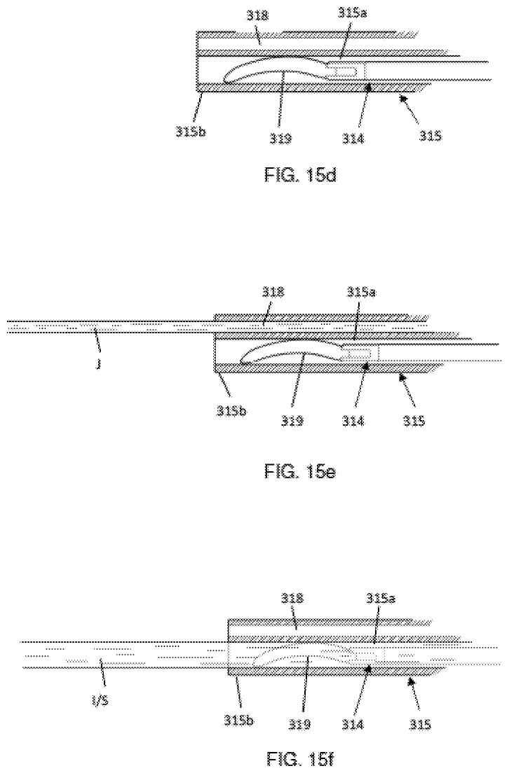

As best seen in FIGS. 15A-15F, the sheath 315 includes a primary lumen 315a sized to slidably receive a shaft of the forceps 314 and a secondary lumen or jet channel 318 adjacent the primary lumen 315a, generally similar to the previous apparatus. The forceps 314 may be movable axially and/or rotatable relative to the sheath 314, e.g., to advance the jaws 319 of the forceps 314 from the distal end 315b of the sheath 315, e.g., as shown in FIGS. 14a-14c and 15a-15c, or retract the jaws 319 into the sheath 315, e.g., as shown in FIGS. 14d-14f and 15d-15f. In addition, similar to the previous apparatus, fluid may be delivered through either of the primary lumen 315a, e.g., a low-pressure fluid flow I/S for irrigation, e.g., as shown in FIGS. 14c, 14f, 15c, 15f, or the jet channel 318, e.g., a high-pressure fluid jet K for hydro-dissection, e.g., as shown in FIGS. 14b, 14e, 15b, 15e.

For example, as shown in FIG. 13b, the housing 316 may include a pair of ports 321a, 321b that communicate with the primary and secondary lumens 315a, 318, respectively, that may be coupled to respective ports on the robotic arm system 330 when the housing 316 is connected to the robotic arm 330. These ports, e.g., including one or more seals and/or connectors (not shown) may then communicate with a source of fluid and/or vacuum, e.g., connected to a proximal end of the robotic arm 330. In addition, as shown, the housing 316 includes a first shaft connector 322 that is coupled to a shaft of the forceps 314, which is connected to a corresponding shaft in the robotic arm system 330. Thus, once connected, the robotic arm shaft may be advanced axially and/or rotated about a longitudinal axis, thereby causing corresponding axial and/or rotational movement of the jaws 319 of the forceps 314.

Optionally, if one or more electrodes or other cautery elements are provided on the jaws 319 (or elsewhere on the distal end of the forceps), the housing 316 may include a connector 323, e.g., an electrical connector, that may be connected to a corresponding connector on the robotic arm system 330 to allow activation of the cautery element(s) during use of the apparatus 320. For example, the robotic arm 330 may be coupled to a generator and/or controller (not shown) for providing electrical or other energy to the cautery element(s). Further optionally, if the sheath 315 and/or forceps 314 include other actuatable features, additional connectors may be provided on the housing 316 and robotic arm system 330. For example, if the forceps 314 includes microfluidic channels and/or sensors, connectors may be provided to allow the sensors to be activated during use.

In another option, as shown in FIG. 15A, the sheath 315 may include a camera and/or other imaging element 324 on the distal end 315b, e.g., to image the jaws 319 and/or otherwise view beyond the distal end 315b of the sheath 315. For example, a CCD, CMOS, or other camera 324 may be provided on the distal end 315b having a field of view FOV sufficient to observe the jaws 319 and/or tissue structures within a surgical space into which the device 320 is introduced. In addition, the imaging element 324 may include one or more LEDs or other light sources (not shown), e.g., necessary to illuminate the field of view FOV. One or more leads 325 may be provided, e.g., embedded within the wall of the sheath 315 or provided in a separate lumen (not shown) that extends to the proximal end of the sheath 315. In this option, one or more additional connectors (not shown) may be provided on the housing 316 to provide power to the imaging element(s) and/or to receive signals from the imaging element(s), e.g., to provide signals to a processor of the control console 340 to generate images on a display (not shown) included in the control console 340.

Unlike the previous apparatus, the surgeon may operate the apparatus 320 to manipulate the sheath 315 remotely as desired, e.g., to introduce the sheath 315 into a surgical space, e.g., through a trocar or other access port (not shown), whereupon the forceps 314 may be deployed and manipulated and/or fluid may be delivered into the surgical space to perform hydro-dissection, irrigation, and/or suction, as needed during the procedure. The robotic arm system 330 may be connected to one or more sources of fluid and/or vacuum, e.g., one or more pumps, one or more power sources, one or more processors and/or controllers, and the like (not shown), which may be activated using the control console 340, similar to conventional robotic surgical procedure systems.

Optionally, any of the devices and apparatus described herein may include one or more additional features. For example, if desired, one or more micro-fluidic channels may be provided on the instrument, e.g., extending to the distal end and/or jaws of the forceps, that may include one or more sensors coupled to a processor (not shown) of the apparatus. Signals from the sensor(s) may be analyzed by the processor, e.g., to identify tissues and/or analyze body fluids to identify the presence of one or more diseases or other conditions.

In addition or alternatively, the devices or apparatus may include a Doppler or other sensor, e.g., carried on one or both jaws of the forceps or other end effector, that may be coupled to a processor to identify blood flow in tissues captured between the jaws. Optionally, the devices or apparatus may include one or more electrodes or other cautery elements, vessel sealing elements, and the like, e.g., on one or both jaws or other location on the end effector, which may be coupled to an energy source (not shown), which may be selectively activated to cauterize tissues that are captured or severed by the forceps or other instrument. One or more actuators (also not shown) may be provided on the handle of the forceps or other location of the apparatus or system, which may be used to activate such sensors and/or cautery elements.

Optionally, any of the devices and systems herein may include one or more imaging elements, e.g., on the distal end of the sheath or instrument, e.g., a CCD, CMOS, or other camera and/or one or more LEDs or light sources (not shown), which may be used to image the end effector of the instrument deployed from the sheath and/or otherwise image a surgical field during a procedure. One or more processors may be coupled to the imaging elements for activating the elements, acquiring images or other signals, and/or for providing output signals to a display, which may be observed by the surgeon during use. For example, a display may be mounted on or otherwise carried on the proximal end of the device, e.g., on the handle 41 of the devices 20, 120 shown in FIG. 5 or 8a, on the proximal end of the sheath 115 shown in FIG. 8a, on the frame 236 shown in FIG. 11a, or separate from the devices or systems, e.g., included in the control console 340 shown in FIG. 13a.

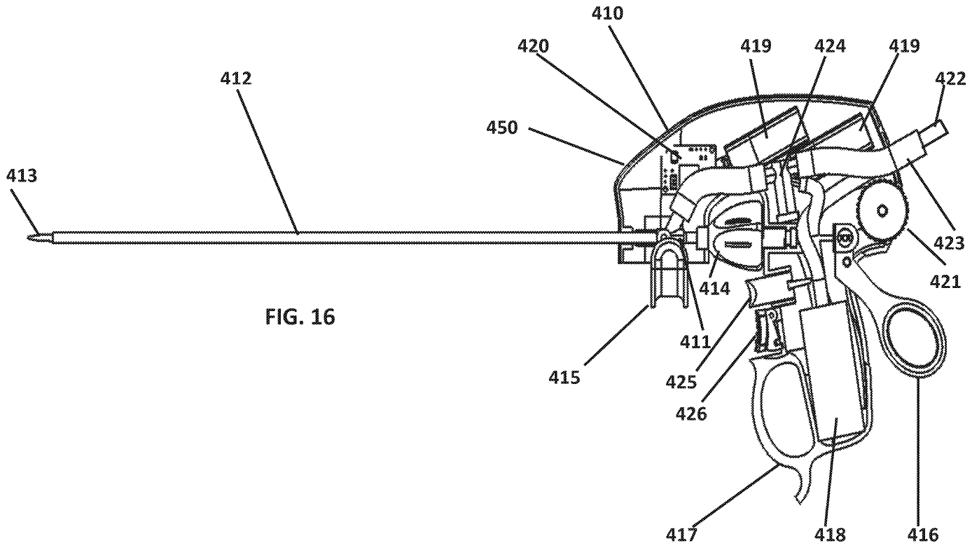

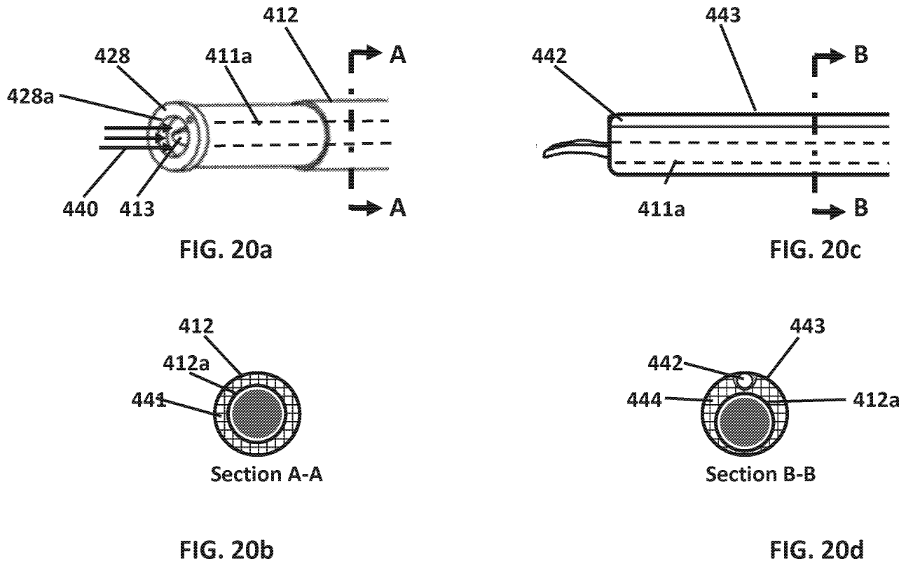

Turning to FIG. 16 another example of a laparoscopic forceps device 410 is shown that may selectively be operated to perform hydro dissection, irrigation, and suction. In the example shown, the device 410 includes a forceps 411 including an elongate shaft 411a including a proximal end coupled to a handle 450 and a distal end carrying a pair of jaws 413. An outer sheath 412 is provided concentrically around the shaft 411 such that the sheath 412 is movable axially relative to the shaft 411a between proximal and distal positions. For example, as shown in FIGS. 20a and 20b, the sheath 412 may include a lumen 412 extending between its distal ends within which the shaft 411a is received. The lumen 412 has an inner diameter larger than the outer diameter of the shaft 411a to provide a fluid flow path through the sheath 412 around the shaft 411a.

In one example, the shaft 411 of a three-millimeter (3 mm) diameter laparoscopic forceps is provided within the length of a five millimeter (5 mm) diameter outer sheath 412, and the jaws 413 of the laparoscopic forceps extend distally beyond a distal tip of the outer sheath 412. Thus, in this example, the handle 450 and sheath 412 may be mounted to a standard forceps instrument to provide the device 410 provided to a surgeon or other user. The handle 450 and sheath 412 may be permanently mounted to the forceps 411 to provide an integral device 410 or may be removably mounted to the forceps 411, e.g., if one or more components of the device 410 are intended to be cleaned and reused. Alternatively, the components of the forceps may be integrally formed together wih the handle 450 and sheath 412.

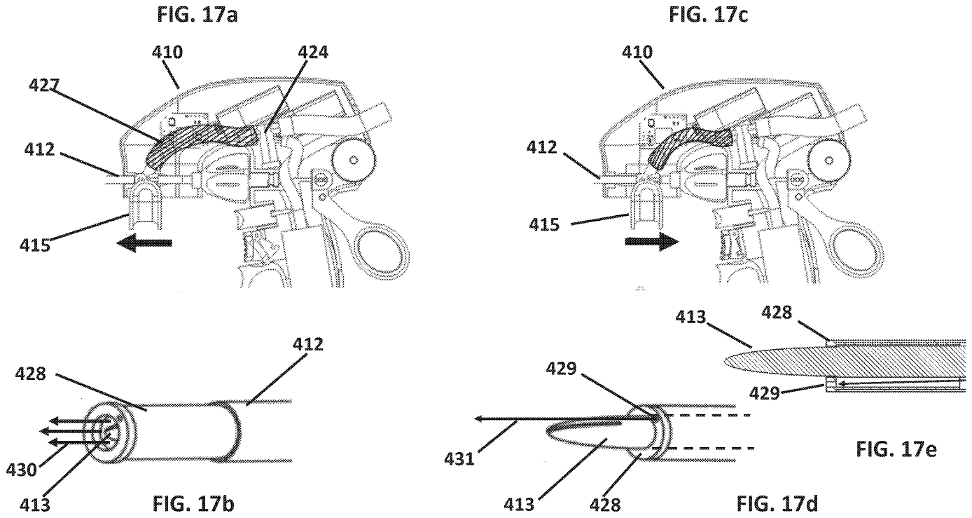

The handle 450 includes one or more actuators for manipulating and/or activating various components of the device 410. For example, the handle 450 may include an actuation knob 414 that allows the surgeon to rotate the shaft 411a and jaws 413 of the forceps 411, e.g., relative to the outer sheath 412. In addition, the handle 450 may include an actuator 415 for manipulating the sheath 412 relative to the forceps 411, e.g., to advance and retract the sheath 412 relative to the jaws 413 of the forceps 411. For example, the sheath 412 may be advanced distally to a distal or advanced position that completely covers the forceps jaws 413, e.g., as shown in FIGS. 17b and 20a, and proximally to a proximal or retracted position where the jaws 413 are exposed, by axial displacement of the sheath actuator 415. The handle 450 may include one or more actuators coupled to the jaws 413 to direct the jaws 413 between open and closed positions. For example, the forceps jaws 413 may be opened and closed via thumb manipulation of a movable ring or other actuator 416, while the stationary loop 417 accommodates the user's third and fourth fingers.

The inner components of the device 410 may be observed in the opened view of the handle housing 450 shown in FIG. 16, i.e., with a cover of the handle removed to facilitate observation of the components. As shown, the device 410 may include a source of pressurized fluid and a power source and/or controller that may be actuated to deliver the pressurized fluid from the device 410, e.g., from the distal tip of the sheath 412, as described further elsewhere herein. In the example shown an electric fluid pump 418 provides a pressurized source of saline fluid, which may be coupled to a reservoir within the handle 450 and/or an external reservoir. Electric power is supplied to the pump 418 via two nine-volt batteries 419 or other power source, with an electronic board 420 maintaining a constant voltage source.

One or more actuators may be provided on the handle 450 to activate the source of pressurized fluid and/or adjust one or more parameters, e.g., pressure, velocity, flow volume of the delivered fluid. For example, an externally accessible control 421 to a potentiometer may be provided to allow the surgeon to vary the voltage input to the pump 418, e.g., to increase or decrease the velocity of the fluid emitted by the device. Fluid input connector 422 accepts an intravenous line connected to an external reservoir e.g., a saline bag (not shown), that is elevated, e.g., approximately one meter above the level of the patient on the operating table. The elevated saline bag provides an additional pressure head supply to the fluid pump 418 to increase the flow velocity of the fluid jet used for surgical hydro dissection. A separate suction connector 423 may be attached to a vacuum line that connects to a standard operating room vacuum source.

Delivery of either fluid flow or vacuum to outer sheath 412 is governed by a mechanical valve 424. Fluid control switch 425 is a spring loaded electrical On/Off switch that powers fluid pump 418. Suction control switch 426 is a mechanical switch that selects vacuum as the mode supplied to outer sheath 412.

FIG. 17a depicts the configuration of the controls in the device 410 when the device 410 is supplying fluid in a low velocity fluid irrigation/suction mode. The sheath actuator 415 is moved forward to the distal position to achieve full distal advancement of the outer sheath 412. A flexible fluid line 427 accommodates forward motion of the actuator 415 to transfer fluid from stationary valve 424 to moveable outer sheath 412. FIG. 17b illustrates the device configuration upon full distal advancement of outer sheath 412. A bushing or other annular member 428 attached to or otherwise provided on the distal end of the outer sheath 412 that is sized to completely cover the forceps jaws 413. Alternatively, the bushing 428 may be integrally formed as part of the sheath 412.

For example, the bushing 428 may include an end cap or distal wall with a distal opening 428a that has a smaller inner diameter than the lumen 412a. The bushing 428 includes one or more relatively small openings or passages, e.g., secondary passage or nozzle 429, adjacent the distal opening 428a that communicate with the lumen 412a and the are distal to the bushing 428. Since the jaws 413 exhibit a distally tapering profile, a large fluid flow path 430 exists between the outer profile of jaws 413 and the inner diameter in the distal opening 428a in the end cap on the bushing 428. This results in low velocity irrigation exiting the distal end of the outer sheath 412 around the jaws 413 with minimal/no fluid flow through the nozzle 420 (given the relatively higher resistance to flow).

FIG. 17c depicts retraction of the outer sheath 412 by backward movement of the sheath actuator 415 to the proximal position, i.e., to configure the device 410 in a hydro-dissection mode. FIG. 17d illustrates the resultant position of the distal end of the device 410, with the forceps jaws 413 fully exposed and functional in opening and closing. The distal opening 428a in the end cap on the distal bushing 428 is sized to contact the shaft of the laparoscopic forceps, preventing circumferential fluid flow from exiting around the periphery of the forceps jaws 413, and forcing all fluid flow to exit through the small diameter nozzle 429 in the end cap of the distal bushing 428, thus forming a high velocity hydro dissection fluid jet. FIG. 17e is a sectional view with an arrow illustrating the resultant fluid path taken during high velocity hydro dissection, as the forceps jaws 413 close off the annular opening in the end cap of the bushing 428 and forces all fluid flow to exit the small diameter nozzle 429. Bushing 428 is constructed of a rigid, non-conductive material able to withstand elevated temperatures exhibited by application of radiofrequency energy to forceps jaws 413 during tissue cautery. Anodized aluminum may be used, as it contains all of the foregoing features.

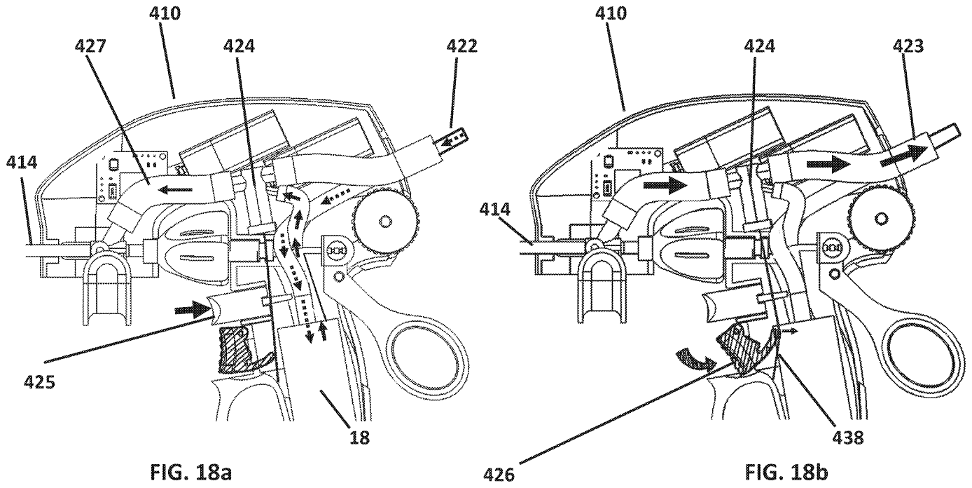

FIG. 18a illustrates activation of fluid flow in the device 410. Button switch 425 is depressed to activate electrical fluid pump 418, causing fluid to flow into the pump 418 via fluid input connector 422, as illustrated by the dashed arrows. Fluid output from the pump 418, as illustrated by the solid arrows, enters valve 424, proceeds through flexible fluid line 427, and continues through the lumen of the sheath 414. FIG. 18b illustrates activation of suction in the device 410. Depression of suction button 426 exerts traction on the inelastic cable 439 that actuates valve 424, simultaneously cutting off antegrade fluid flow while enabling retrograde suction to occur. The solid arrows illustrate the path of suction flow in a retrograde manner from the lumen of outer sheath 414 through valve 424 to the suction connector 423.

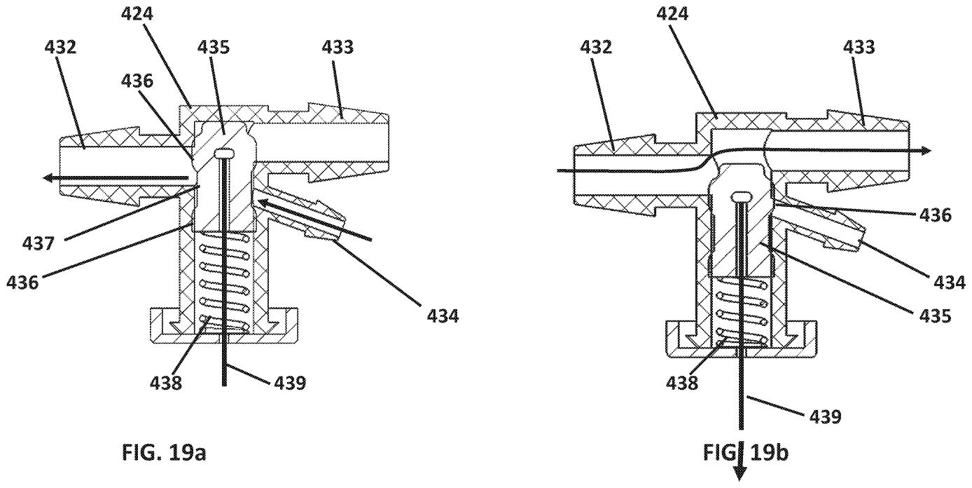

FIG. 19a depicts the fluid and suction control valve 424 in its normal resting position, in which suction is cut off, but fluid flow is enabled. The valve stem 435 contains an hourglass shape, with wide portions 436 and a central narrow portion 437. Compression spring 438 exerts force on valve stem 435, displacing it superiorly such that the enlarged portion 436 completely obstructs vacuum flow between valve outlet port 432 and suction inlet port 433.

In the resting position, the narrow valve stem portion 437 is aligned with fluid intake port 434 and valve outlet port 432, enabling fluid flow to occur through the control valve 424. In the normal resting position of valve 424, no tension is exerted on valve stem 435 by the attached inelastic cable 439. In one example, the valve stem 435 may be constructed of an inelastic polymer such as Nylon, polyethylene, or polytetrafluoroethylene (PTFE); or it may be constructed of a partially elastic polymer such as polyurethane.

FIG. 19b depicts control valve 424 activated to supply suction while cutting off fluid flow. Upon exertion of traction to the cable 439, the spring 438 is compressed and the valve stem 435 is pulled into position whereby it occludes fluid flow from the fluid intake port 434 while opening a path from the suction intake port 433 to the valve outlet port 432.

FIG. 20a shows the distal portion of the device 410 in the suction configuration, with the dotted outline representing the shaft of the laparoscopic forceps 411 inside the outer sheath 412. Suction airflow 440 occurs in the annular space between the forceps jaws 412 and the opening in the end cap of the bushing 428. FIG. 20b is a sectional view of the instrument shaft at A-A, with the cross-hatched region 441 depicting the suction flow path area between the instrument shaft 411 and the outer sheath 412.

FIG. 20c shows the distal portion of a previous device described in one of the applications incorporated by reference herein, with a small diameter stainless steel tube 442 welded to the inner aspect of outer tube 443. The dotted outline represents the shaft of the laparoscopic forceps 411 inside the outer tube 443. FIG. 20d is a sectional view of the instrument shaft at B-B, with the cross-hatched region 444 depicting the suction flow path area between the instrument shaft 411 and the contours of the jet flow tube 442 and the outer tube 443, for an instrument with the previous configuration. Calculations show that the cross-sectional suction flow area 444 of the previous device is 22.2% less than the effective flow area 441 of the proposed device. This suction capability will be significant during clinical use, when blood clots and debris are encountered during suction use.

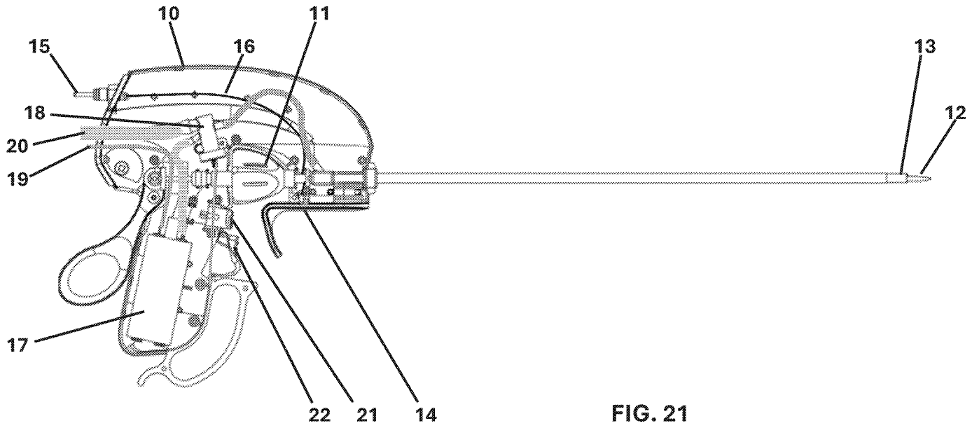

Turning to FIG. 21, another exemplary instrument 510 is shown that generally includes a laparoscopic forceps 511 including an elongate shaft including a proximal end coupled to a handle 550 and a distal end carrying a pair of jaws 512. An outer sheath 513 is provided concentrically around the shaft such that the sheath 513 is movable axially relative to the forceps 511, e.g., all of which may be constructed and/or manipulated generally similar to the device 410 shown in FIG. 16. For example, a bushing or other annular member 528 may be provided on the distal end of the sheath 512 that includes a primary distal opening 528a and a secondary opening or nozzle 529, similar to the device 410.

The sheath 512 may be movable axially relative to the forceps 511, e.g., using an actuator 515 to move the sheath 512 between positions or configurations that allow one or more of hydro dissection, irrigation, suction, and electrocautery capability. In one example, a three millimeter (3 mm) diameter laparoscopic forceps 511 may be incorporated into the device 510, and the forceps jaws 512 lie within or adjacent a lumen of the coaxial sheath 512. Advancement of the coaxial sheath 512 to cover the forceps jaws 513 is performed via manipulation of a sliding actuator 515 on the handle 550 of device 510. The sliding actuator 515 may also be configured to operate an electrical switch that supplies electrocautery energy to the forceps jaws 513. Electrical connectors 523 allows attachment of a cable leading to an electrosurgical generator (not shown). One or more wires, cables, or other conducts, e.g., conducting cable 516 inside the handle 550 of device 510, may conduct electrocautery current from the connector 523 (and the generator coupled to the connector 523) to the laparoscopic forceps 511.

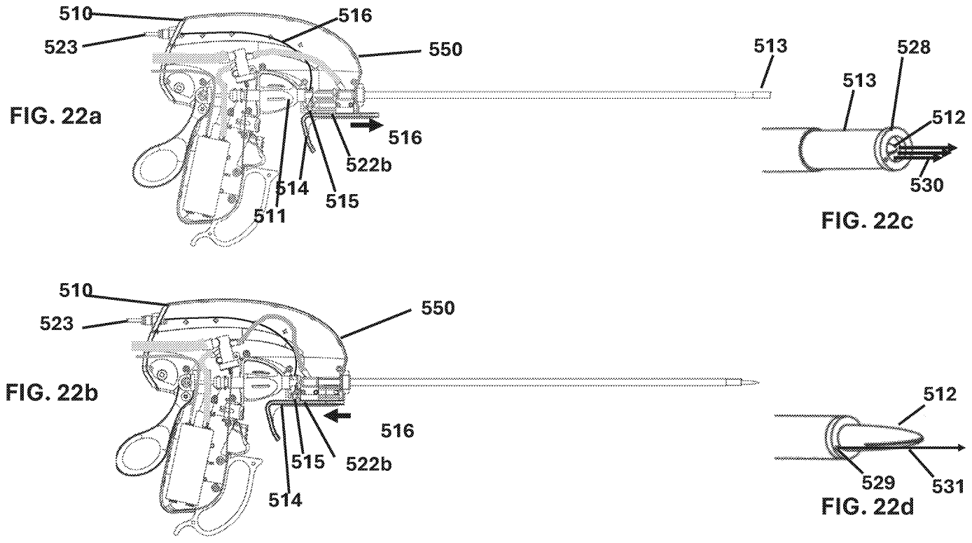

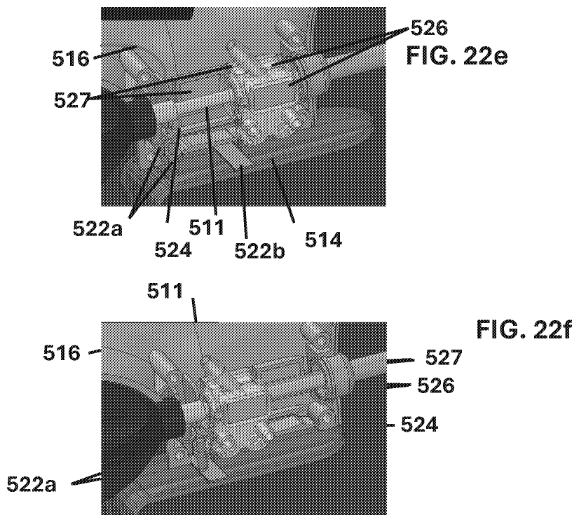

FIGS. 22a and 22b shown additional components that may be provided within the handle 550 (visible in the drawings since a portion of the handle 550 has been removed). For example, a source of pressurized fluid, e.g., a fluid pump 517, may be provided that supplies pressurized fluid to a control valve 518, which controls delivery of either fluid flow or vacuum suction to the coaxial sheath 512. Vacuum supply into the control valve 518 occurs via a suction connection 520, while fluid supply to pump 517 is performed via fluid connector 519 that leads to reservoir, e.g., an intravenous saline bag elevated above the surgical field or other external reservoir (not shown) that may be connected to the connector 519. One or more actuators may be provided on the handle 550 for controlling the valve 518 and/or the sources of pressurized fluid and suction. For example, the handle 550 may include a fluid control button 521 that is depressed to deliver fluid delivery out of the distal end of coaxial sheath 512, and suction control button 522 that is depressed to provide vacuum suction to the device tip.

FIG. 22a depicts the elements associated with supplying and controlling electrocautery energy to the forceps jaws 512. Current from the electrosurgical generator is delivered to the jaws 513 of the forceps 511 via the electrical connector 523F and one or more insulated conducting cables 516. The sliding actuator 515 controls extension and retraction of the coaxial sheath 51 to retract or expose the jaws 513 of the forceps 511, while simultaneously operating an electrical switch to interrupt or supply electrocautery energy to the jaws 513. In the example shown in FIGS. 22e and 22f, the electrical switch includes a conductive plate electrode 522b attached to the sliding actuator 515, and one or more stationary post electrodes 522a fixed to the inside handle surface of device 510. FIG. 22e is a magnified two-dimensional view of the electrical switch mechanism, with the plate electrode 522b attached to the sliding actuator 515. The sliding actuator 515 is advanced distally in this illustration, such that plate electrode 522b does not contact the (in this case two) post electrodes 522a. The conducting cable 516 connects to the left-sided post electrode 522a, while insulated cable 524 connects the right-sided post electrode 522a to the shaft of laparoscopic forceps 511. Thus, with the sheath 512 advanced to cover the jaws 513, the circuit is opened, and no electrocautery current is capable of being delivered to the jaws 513 in the distal position. In addition or alternatively, an actuator (not shown) may be provided on the handle that may be activated to manually open and close the circuit, if desired.

Optionally, the device 510 may include one or more features to stabilize the sheath 512 during manipulation and/or to provide feedback to the operator confirming the position of the sheath 512. In the example shown in FIGS. 22e and 22F, actuator attached magnets 526 are attached to the sliding actuator 515 that interact with two handle attached magnets 527, attached to the inner surface of the handle 550. The attractive force between the actuator magnets 526 and the distal handle magnet 527 stabilizes the coaxial sheath 512 when advanced to the distal position to cover the forceps jaws 512. FIG. 22c illustrates the configuration of the tip of device 510 when the sliding actuator 515 is advanced distally and the coaxial sheath 512 covers the forceps jaws 513. Ample clearance between the inner surface of the primary opening 528a of the bushing 528 and the outer surface of forceps jaws 513 results in the delivery of low velocity fluid irrigation 530.

FIG. 22b illustrates the device 510 with the sliding actuator 515 retracted proximally, such that both the left and right post electrodes 522a contact the plate electrode 522b. The magnified two-dimensional view in FIG. 22f shows that electrocautery current may now be conducted to the jaws 513 of the forceps 511 via the electrical cables 516, 524. The actuator attached magnets 526 are in attractive coupling with the proximal handle attached magnet 527, stabilizing the coaxial sheath 512 in the retracted or proximal position. When the sliding actuator 515 is moved from the advanced position to the retracted position, the shifting position of actuator attached magnets 526 from the distal to the proximal handle attached magnets 527 may cause a clicking sound and/or movement of the sliding actuator 515 may cause a vibration or other perceptible movement, which may provide the surgeon with aural and/or haptic feedback that the coaxial sheath 512 has been fully retracted to the proximal position. FIG. 22f illustrates that with complete retraction of the coaxial sheath 512 to the proximal position, the inner surface of the primary opening 528a in the bushing 528 seals against the outer surface of laparoscopic forceps 511 immediately proximal to the jaws 513. Thus, subsequent delivery of pressurized fluid forces all fluid flow to exit from the nozzle, thereby forming a high velocity jet 531 to perform surgical hydro dissection.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A device for performing a surgical procedure within a patient's body, comprising:
a tubular member comprising a proximal end, a distal end, and a lumen extending between the proximal and distal ends;
a source of fluid coupled to the lumen to deliver pressurized fluid through the lumen;
an instrument comprising a shaft slidably received within the lumen and an end effector on an end of the shaft disposed adjacent the distal end of the tubular member, the shaft sized to accommodate fluid flowing from the source through the lumen around the shaft;
a handle comprising a first actuator coupled to the tubular member to move the tubular member between a distal position where the distal end covers the end effector and a proximal position where the end effector is exposed beyond the distal end; and
an annular member on the distal end including a primary passage therethrough sized such that, in the distal position, fluid from the source passes from the lumen around the end effector to deliver the fluid out the primary passage, and, in the proximal position, the annular member engages the shaft to seal the primary passage, the annular member comprising a secondary lumen adjacent the primary passage having a cross-section smaller than the primary passage such that the fluid from the source flows through the lumen and out the secondary passage to provide hydro-dissection fluid.

2. The device of claim 1, further comprising a second actuator on the handle for actuating the end effector.

3. The device of claim 2, wherein the end effector comprises a pair of jaws coupled to the second actuator for directing the jaws between open and closed positions.

4. The device of claim 3, wherein the jaws comprise substantially blunt contact surfaces configured to grasp tissue between the jaws in the closed position.

5. The device of claim 4, further comprising a third actuator on the handle configured to activate the source of fluid after the jaws grasp tissue to deliver the pressurized fluid through the lumen with the tubular member in the proximal position to perform hydro-dissection.

6. The device of claim 1, wherein the source of fluid comprises a pump carried by the handle.

7. The device of claim 6, wherein the source of fluid further comprises a reservoir of fluid coupled to the pump.

8. The device of claim 7, wherein the reservoir comprises a bag of fluid external to the device.

9. The device of claim 8, further comprising tubing coupled between the bag and the pump.

10. The device of claim 1, further comprising:
a connector on the handle configured to be coupled to a source of vacuum;
an actuator for selectively opening a first flow path between the source of pressurized fluid and the lumen and a second path between the connector and the lumen.

11. The device of claim 10, wherein the actuator comprises an actuatable valve configured to selectively open and close the first and second flow paths.

12. The device of claim 11, wherein the valve is biased to a position wherein the first path is open to deliver pressurized fluid to the lumen and the second path is closed.

13. The device of claim 12, wherein the actuator is configured to overcome the bias to close the first path and open the second path to deliver the vacuum through the lumen to provide suction into the primary opening in the annular member with the tubular member in the distal position.

14. The device of claim 1, further comprising a cautery element on the end effector, the handle comprising comprises a connector for coupling the cautery element to a source of energy to deliver energy to the cautery element to cauterize tissue contacted by the cautery element.

15. The device of claim 14, wherein the cautery element comprises one or more electrodes on the end effector.

16. The device of claim 14, wherein the end effector comprises a pair of jaws and wherein the cautery element comprises one of a conductive surface and an electrode on one of the jaws.

17. The device of claim 14, wherein the source of electrical power comprises a generator configured to be coupled to the connector to deliver electrical energy to the cautery element via the one or more leads to cauterize tissue contacted by the cautery element.

18. The device of claim 14, further comprising a switch configured to open and close a conductive path from the connector to the cautery element.

19. The device of claim 18, wherein the switch is configured to close the conductive path when the tubular member is positioned in the proximal position such that the end effector is fully exposed from the distal end to allow deliver of electrical energy of the cautery element.

20. The device of claim 18, wherein the switch is configured to open the conductive path when the tubular member is moved distally from the proximal position to prevent deliver of electrical energy of the cautery element when the end effector is partially or completed covered by the distal end.

21. The device of claim 18, wherein the first actuator comprises a sliding actuator configured to be manipulated to move the tubular member between the proximal and distal positions, and wherein the switch is coupled to the sliding actuator.

22. The device of claim 18, wherein the switch comprises a stabilizer that stabilizes the switch in positions that open and close the conductive path.

23. The device of claim 22, wherein the stabilizer comprises a magnetic coupling.

24. The device of claim 22, wherein the stabilizer is configured to provide haptic feedback to a user when the tubular member is in the proximal position to provide confirmation that the conductive path is closed.

25-33. (canceled)

34. A method for performing a surgical procedure within a patient's body, comprising:
providing a laparoscopic device comprising a tubular member comprising a proximal end, a distal end, and a lumen extending between the proximal and distal ends; an instrument comprising a shaft slidably received within the lumen and an end effector on an end of the shaft disposed adjacent the distal end of the tubular member; a handle comprising a first actuator coupled to the tubular member to move the tubular member between a distal position where the distal end covers the end effector and a proximal position where the end effector is exposed beyond the distal end; and an annular member on the distal end including a primary passage therethrough and a secondary lumen adjacent the primary passage having a cross-section smaller than the primary passage;
introducing the distal end into a patient's body;
with the tubular member in the proximal position such that the primary passage is sealed against the shaft, delivering pressurized fluid through the lumen such that the fluid flows through the lumen and out the secondary passage to provide hydro-dissection fluid to dissect tissue within the patient's body; and
with the tubular member in the distal position, delivering pressurized fluid through the lumen around the shaft and out the primary passage to provide irrigation fluid.

35. (canceled)
